(19) United States
(12) Patent Application Publication
(10) Pub. No.: US 2024/0212292 A1
(43) Pub. Date: Jun. 27, 2024

(54) AUGMENTED REALITY PATIENT ASSESSMENT MODULE

(71) Applicants: Zimmer US, Inc., Warsaw, IN (US); ZIMMER, INC., Warsaw, IN (US)

(72) Inventors: Ted Spooner, Grand Rapids, MI (US); Dave Van Andel, Spring Lake, MI (US)

(21) Appl. No.: 18/557,249

(22) PCT Filed: Apr. 27, 2022
(86) PCT No.: PCT/US2022/026509

§ 371 Date: Oct. 25, 2023

Related U.S. Application Data
(60) Provisional application No. 63/180,456, filed on Apr. 27, 2021.
(60) Provisional application No. 63/303,683, filed on Jan. 27, 2022.

(51) Int. Cl. G06T 19/00; A61B 5/00; A61B 5/11; A61B 34/10

(52) U.S. Cl. CPC G06T 19/006; A61B 5/11; A61B 5/4528; A61B 5/4538; A61B 5/743; A61B 34/10; A61B 2034/105

(57) ABSTRACT

Patient mobility assessments may be improved using an augmented reality patient assessment module. To reduce subjectivity of a mobility assessment, a depth camera may be used to determine precise patient motion, generate a skeletal model of the patient, and determine range of motion for various patient joints. An augmented reality head-mounted display may include a transparent display screen, and may be used to display the skeletal model overlaid on the patient while the patient is being viewed through the transparent display screen. A medical practitioner may guide the patient through a series of musculoskeletal evaluation activities, which may be used to generate the skeletal model of the patient and determine range of motion for various patient joints. The skeletal model and range of motion information may be used to generate a predicted postoperative skeletal model, which may indicate an improved range of motion based on a surgical procedure.

### CLAIM OF PRIORITY

This application claims the benefit of U.S. Provisional Patent Application Ser. No. 63/180,456, filed on Apr. 27, 2021, and also claims the benefit of U.S. Provisional Patent Application Ser. No. 63/303,683, filed on Jan. 27, 2022, the benefit of priority of each of which is claimed hereby, and each of which is incorporated by reference herein in its entirety.

### FIELD

The present application relates to patient mobility assessment using augmented reality.

### BACKGROUND

A patient's mobility may be affected by various changes in the patient's musculoskeletal system. For example, the range of motion of a patient joint may be decreased by arthritis or due to an injury. When conducting assessments of patient mobility, a medical practitioner typically guides a patient through a series of exercises to determine range of motion. However, the patient range of motion is often a subjective determination made by the medical practitioner.

Diagnostics are used to evaluate a patient to determine whether the patient needs a surgical procedure, such as for upper extremities (e.g., a shoulder or elbow), lower extremities (e.g., knee, hip, etc.), or the like. These procedures are performed hundreds of thousands of times a year in the United States. Surgical advancements have allowed surgeons to use preoperative planning, display devices, and imaging, to improve diagnoses and surgical outcomes.

An augmented reality (AR) or mixed reality (MR) device (AR and MR being used interchangeably) allows a user to view displayed virtual objects that appear to be projected into the real environment, which is also visible. AR devices typically include two display lenses or screens, including one for each eye of a user. Light is permitted to pass through the two display lenses such that aspects of the real environment are visible while also projecting light to make virtual elements visible to the user of the AR device.

### BRIEF DESCRIPTION OF THE DRAWINGS

FIGS. 1A-1B are diagrams of a skeletal modelling system, in accordance with some embodiments.

FIGS. 2A-2B are diagrams of a skeletal motion modelling system, in accordance with some embodiments.

FIG. 3 is a diagram of a remote skeletal modelling system, in accordance with some embodiments.

FIG. 4 is a diagram of an augmented reality joint viewing system, in accordance with some embodiments.

FIGS. 5A-5B are diagrams of a spherical skeletal modelling system, in accordance with some embodiments.

FIG. 6 illustrates a flow chart showing an augmented reality patient assessment method, in accordance with some embodiments.

FIG. 7 illustrates a user interface for selecting an application, in accordance with some embodiments.

FIG. 8 illustrates a first user interface for patient selection, in accordance with some embodiments.

FIG. 9 illustrates a second user interface for patient selection, in accordance with some embodiments.

FIG. 10 illustrates a user interface for selecting a patient, in accordance with some embodiments.

FIG. 11 illustrates a user interface for displaying patient information, in accordance with some embodiments.

FIG. 12 illustrates assessment user interface for selecting an assessment, in accordance with some embodiments.

FIG. 13 illustrates user interfaces for displaying aspects of an AR assessment, in accordance with some embodiments.

FIG. 14 illustrates a user interface and component for displaying aspects of an AR assessment and skeletal overlay, in accordance with some embodiments.

FIG. 15 illustrates a user interface and component for displaying aspects of an AR assessment and skeletal overlay, in accordance with some embodiments.

FIG. 16 illustrates a user interface for displaying results of an AR assessment, in accordance with some embodiments.

FIG. 17 illustrates user interfaces for selecting a surgical demonstration in augmented reality, in accordance with some embodiments.

FIG. 18 illustrates a user interface for selecting a surgical demonstration in augmented reality, in accordance with some embodiments.

FIG. 19 illustrates a user interface and component for displaying aspects of an AR demonstration and 3D bone model, in accordance with some embodiments.

FIG. 20 illustrates an example 3D bone model AR view, in accordance with some embodiments.

FIG. 21 illustrates an example 3D bone model in an AR view, in accordance with some embodiments.

FIG. 22 illustrates a flowchart showing a technique for performing a patient assessment using augmented reality in accordance with at least one example of this disclosure.

FIG. 23 illustrates a flowchart showing a technique for displaying a surgical demonstration using augmented reality in accordance with at least one example of this disclosure.

FIG. 24 illustrates an example of a block diagram block diagram of a machine upon which any one or more of the techniques discussed herein may perform in accordance with some embodiments.

### DETAILED DESCRIPTION

The present disclosure describes technical solutions to technical problems facing patient mobility assessments. To reduce subjectivity of a mobility assessment, a depth camera (e.g., depth sensor) may be used to determine precise patient motion, generate a skeletal model of the patient, and determine range of motion for various patient joints. An AR or MR head-mounted display (HMD) may include a transparent display screen, and may be used to display the skeletal model overlaid on the patient while the patient is being viewed through the transparent display screen.

A medical practitioner may guide the patient through a series of musculoskeletal evaluation activities. Depth sensor information captured during the evaluation activities may be used to generate the skeletal model of the patient and determine range of motion for various patient joints. The evaluation activities may be displayed on the HMD while the patient is being viewed through the transparent display screen. The display of the evaluation activities may include an indication of the patient's current range of motion for one or more joints.

The skeletal model and range of motion information may be used to generate a predicted postoperative skeletal model. The predicted postoperative skeletal model may indicate an improved range of motion based on a surgical procedure. For example, a femoroacetabular impingement may limit hip joint mobility, and the predicted postoperative skeletal model may indicate an improved hip range of motion based on an acetabular resurfacing procedure. The evaluation activities may be used to identify one or more surgical procedures that may improve joint range of motion. For example, a hip flexion and extension evaluation activity may indicate a reduced hip range of motion, and an acetabular resurfacing procedure or other hip procedures may be suggested to the medical practitioner to improve hip range of motion. The predicted postoperative skeletal model may be output for display, and may be overlaid on the patient while the patient is being viewed through the transparent display screen. The predicted postoperative skeletal model may be provided to a patient viewing device, such as a patient HMD, tablet, or other display device.

Additional musculoskeletal evaluation activities may be used to reassess patient mobility, such as following a surgical procedure. The postoperative evaluation activities may be used to gather postoperative depth sensor data and generate a postoperative skeletal model. This postoperative skeletal model may be compared to the preoperative skeletal model, such as by displaying the postoperative model superimposed on the preoperative model. One or both of the preoperative model and postoperative model may be superimposed on the user, such as superimposing both models on the patient while the patient is being viewed through a transparent HMD screen.

An optical camera (e.g., image capture device) may capture images (e.g., still images or motion video), such as during preoperative or postoperative assessment. The captured images may be stored with associated preoperative or postoperative skeletal models, and may be used by the medical practitioner or patient to view the skeletal model overlaid on the patient. The captured images may allow the medical practitioner or patient to view a particular joint position (e.g., full flexion, full extension) or view a video of the patient's current range of motion for a joint.

Systems and methods described herein may be used for evaluating a patient before, during, or after completion of an orthopedic surgery on a portion of a body part of the patient. The orthopedic surgery may include a joint repair, replacement, revision, or the like. The evaluation of a patient is an important pre-, intra-, and post-surgical aspect of the treatment journey. Range of motion information or quality of motion information, in particular may be helpful for knowing a patient's limitations pre-intervention, a degree of repair intra-operatively, and recovery progression post-intervention.

Systems and methods described herein may be used to display, in augmented or virtual reality, a feature, a user interface, a component (e.g., a three-dimensional (3D) model, an overlay, etc.), or the like. A 3D model may include a bone model, such as a general bone model or a patient-specific bone model (e.g., generated from patient imaging). An overlay may include a skeletal overlay, for example a set of joints and segments connecting the joints representing patient joints and bones or other anatomy. The overlay may be displayed overlaid on a patient (e.g., with the overlay virtually displayed in an augmented or mixed reality system with the patient visible in the real world).

An augmented reality (AR) device allows a user to view displayed virtual objects that appear to be projected into the real environment, which is also visible. AR devices typically include two display lenses or screens, including one for each eye of a user. Light is permitted to pass through the two display lenses such that aspects of the real environment are visible while also projecting light to make virtual elements visible to the user of the AR device.

Augmented reality is a technology for displaying virtual or “augmented” objects or visual effects overlaid on a real environment. The real environment may include a room or specific area, or may be more general to include the world at large. The virtual aspects overlaid on the real environment may be represented as anchored or in a set position relative to one or more aspects of the real environment. For example, a virtual object such as a menu or model may be configured to appear to be resting on a table. An AR system may present virtual aspects that are fixed to a real object without regard to a perspective of a viewer or viewers of the AR system. For example, a virtual object may exist in a room, visible to a viewer of the AR system within the room and not visible to a viewer of the AR system outside the room. The virtual object in the room may be displayed to the viewer outside the room when the viewer enters the room. In this example, the room may function as a real object that the virtual object is fixed to in the AR system.

An AR device may include one or more screens, such as a single screen or two screens (e.g., one per eye of a user). The screens may allow light to pass through the screens such that aspects of the real environment are visible while displaying a virtual object. The virtual object may be made visible to a wearer of the AR device by projecting light. The virtual object may appear to have a degree of transparency or may be opaque (i.e., blocking aspects of the real environment).

An AR system may be viewable to one or more viewers, and may include differences among views available for the one or more viewers while retaining some aspects as universal among the views. For example, a heads-up display may change between two views while virtual objects may be fixed to a real object or area in both views. Aspects such as a color of an object, lighting, or other changes may be made among the views without changing a fixed position of at least one virtual object.

A user may see a virtual object presented in an AR system as opaque or as including some level of transparency. In an example, the user may interact with the virtual object, such as by moving the virtual object from a first position to a second position, or selecting an indication (e.g., on a menu). For example, the user may move or select an object with a gesture or hand placement. This may be done in the AR system virtually by determining that the hand has moved into a position coincident or adjacent to the object (e.g., using one or more cameras, which may be mounted on an AR device, and which may be static or may be controlled to move), and causing the object to move or respond accordingly. Virtual aspects may include virtual representations of real-world objects or may include visual effects, such as lighting effects, etc. The AR system may include rules to govern the behavior of virtual objects, such as subjecting a virtual object to gravity or friction, or may include other predefined rules that defy real world physical constraints (e.g., floating objects, perpetual motion, etc.). An AR device may include a camera on the AR device. The AR device camera may include an infrared camera, an infrared filter, a visible light filter, a plurality of cameras, a depth camera, etc. The AR device may project virtual items over a representation of a real environment, which may be viewed by a user.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

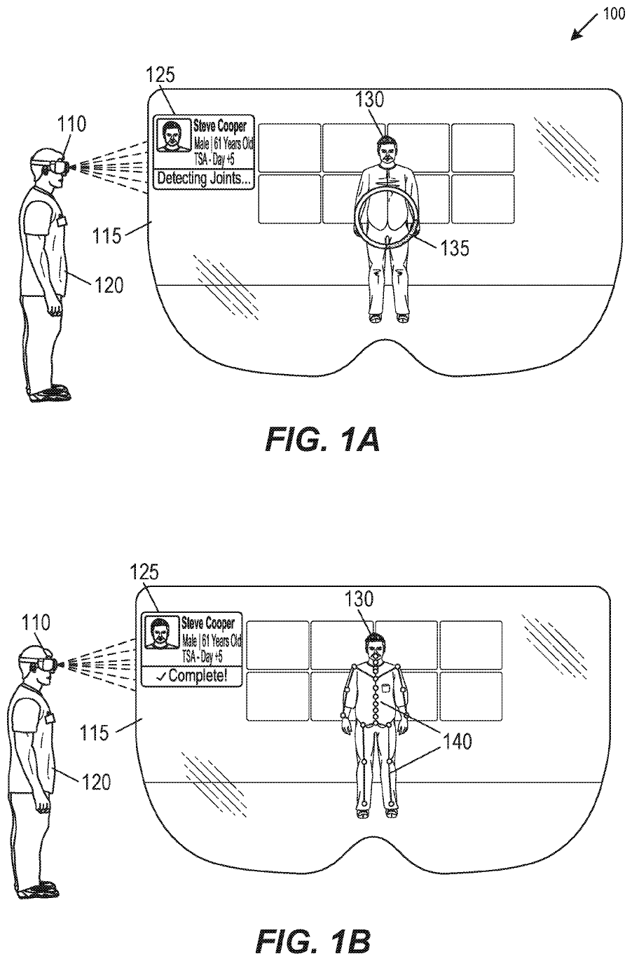

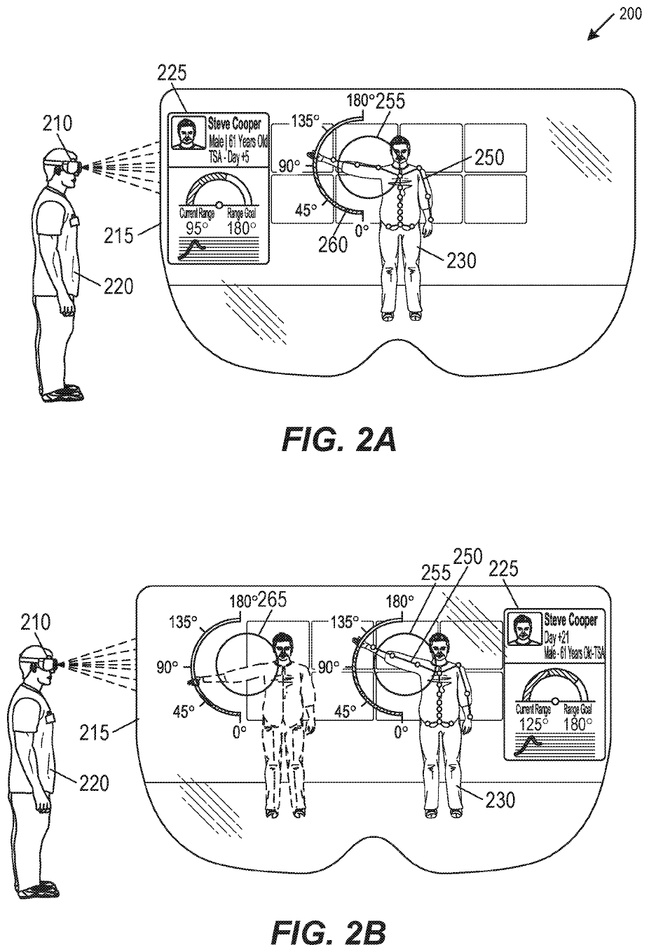

FIGS. 1A-1B are diagrams of a skeletal modelling system 100, in accordance with some embodiments. System 100 may include an HMD 110 with an AR display 115. The HMD 110 may be worn by a medical practitioner 120, and may be used to display patient information 125 while viewing the patient 130 through the display 115. The HMD 110 may use depth sensors, image sensors, and other sensors to identify the patient 130. As shown in FIG. 1A, the HMD 110 may superimpose a circle 135 to indicate that the patient has been identified. The HMD 110 may use one or more of the depth sensors, image sensors, and other sensors to generate a preoperative skeletal model. The preoperative skeletal model may be generated based on a stationary or moving patient. As shown in FIG. 1B, the HMD 110 may superimpose a preoperative skeletal model 140 over the patient 130. In an embodiment, a generalized initial preoperative skeletal model is generated based on a stationary view of the patient, and the preoperative skeletal model is updated continually based on additional depth and image sensor data. The model may be updated based on a series of musculoskeletal evaluation activities, such as shown in FIGS. 2A-2B.

FIGS. 2A-2B are diagrams of a skeletal motion modelling system 200, in accordance with some embodiments. System 200 may include an HMD 210 with an AR display 215. The HMD 210 may be worn by medical practitioner 220, and may be used to display range of motion and other patient information 225 while viewing the patient 230 through the display 215. The medical practitioner 220 may guide the patient 230 through a series of musculoskeletal evaluation activities. The evaluation activities may be used to update the skeletal model 250 of the patient 230. As shown in FIG. 2A, the evaluation activities may be used to determine a shoulder joint range of motion 255. The HMD 210 may display information about the range of motion, such as a range of motion angle indication 260 superimposed on the skeletal model 250 and the patient 230. The HMD 210 may also display current and historical range of motion information within the patient information 225.

The skeletal model 250 and range of motion information may be used to generate a predicted postoperative skeletal model with an associated improved range of motion based on a surgical procedure. System 200 may identify a reduced range of motion for a shoulder joint, and may generate a predicted postoperative skeletal model with an improved shoulder range of motion. As shown in FIG. 2B, HMD 210 may display the reduced shoulder joint range of motion 265 and the predicted postoperative range of motion 255. The reduced shoulder joint range of motion 265 may be displayed in a dashed-line or semi-transparent format to differentiate it from the predicted postoperative range of motion 255. In an embodiment, system 200 may suggest an acromioclavicular resurfacing procedure or other shoulder procedure to improve shoulder range of motion. System 200 may present the medical practitioner 220 with one or more surgical procedures for selection, and the predicted postoperative range of motion 255 may be generated based on one or more selected procedures.

Following a surgical procedure, subsequent musculoskeletal evaluation activities may be used to reassess skeletal model 250 and range of motion information. The postoperative evaluation activities may be used to gather postoperative depth sensor data and generate a postoperative skeletal model. Similar to FIG. 2B, HMD 210 may display a pair of patient skeletal models, such as displaying a preoperative reduced shoulder joint range of motion next to a postoperative range of motion.

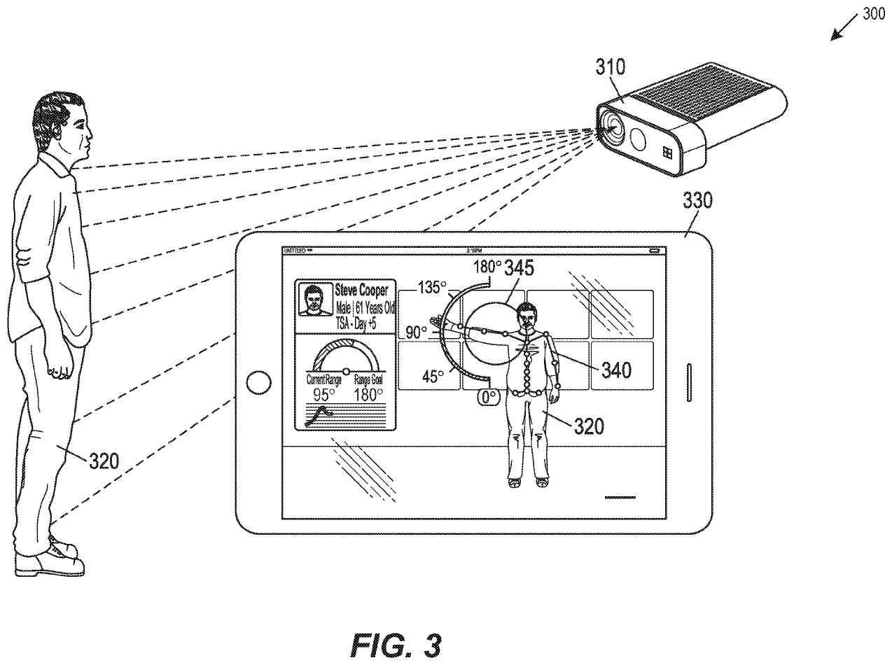

FIG. 3 is a diagram of a remote skeletal modelling system 300, in accordance with some embodiments. System 300 may include a skeletal modeling device 310, which may include depth sensors, image sensors, and other sensors to generate a skeletal model 340 of patient 320. A display device 330 may be used to display the skeletal model 340 and a joint range of motion 345 overlaid on a captured image of the patient 320. Images and depth sensor data captured by skeletal modeling device 310 may be stored with associated preoperative or postoperative skeletal models, and may be used by the medical practitioner or patient to view images or video of the skeletal model 340 overlaid on the patient 320. In an embodiment, the skeletal modeling device 310 may be used within a patient's home or other location remote from a medical practitioner. This may improve remote assessment, such as by enabling the patient 320 to participate in preoperative or postoperative musculoskeletal evaluation activities without requiring a visit to the medical practitioner.

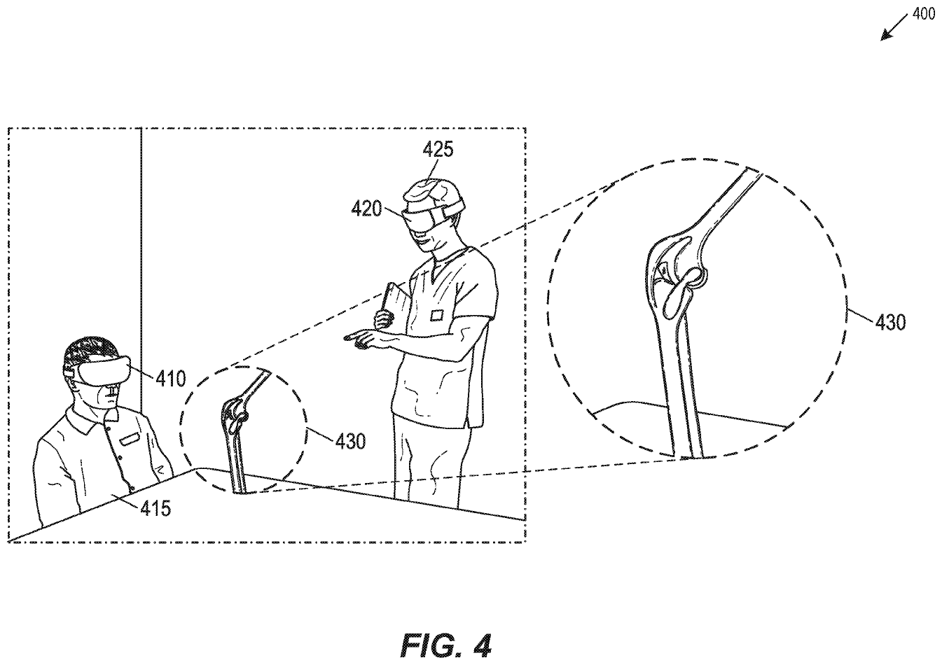

FIG. 4 is a diagram of an AR joint viewing system 400, in accordance with some embodiments. System 400 may include one or more AR displays, such as a patient AR display 410 worn by a patient 415 and a practitioner AR display 420 worn by a medical practitioner 425. The medical practitioner 425 may use system 400 to demonstrate preoperative or postoperative joint mobility. For example, medical practitioner 425 may display an AR image of the pre-operative knee state within both the patient AR display 410 and the practitioner AR display 420. The AR image may be anchored to a position on a table or to another real-world position. The medical practitioner 425 may interact with the AR image, such as by causing one or more bones or soft tissues to be highlighted to aid in describing the pre-operative knee state. System 400 may be used to display and discuss one or more skeletal models, such as a preoperative skeletal model, a predicted postoperative skeletal model, or a postoperative skeletal model.

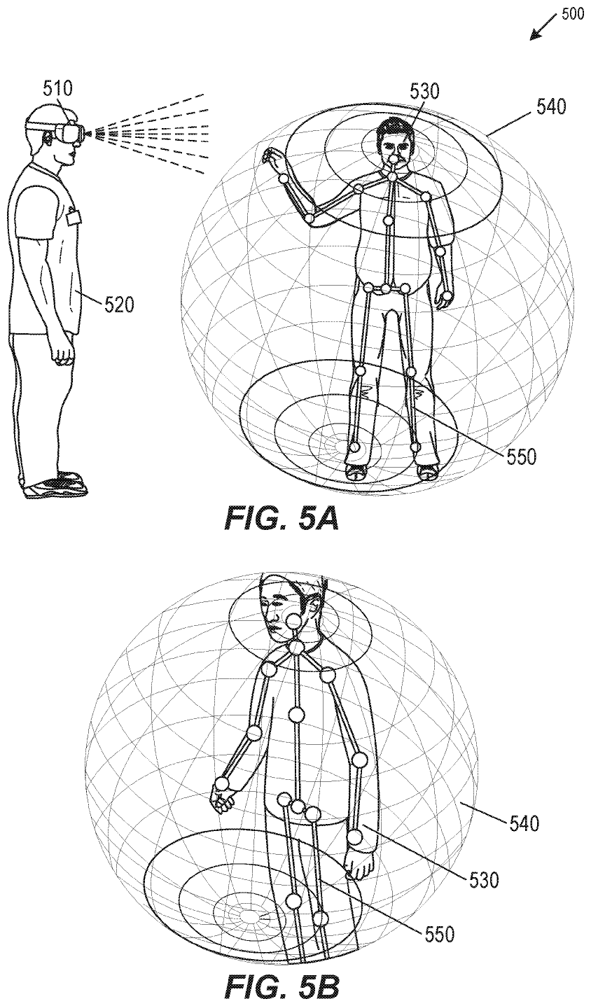

FIGS. 5A-5B are diagrams of a spherical skeletal modelling system 500, in accordance with some embodiments. System 500 may include an AR HMD 510 worn by a medical practitioner 520 while viewing a patient 530. System 500 may display an assessment sphere 540 displayed around the patient 530 while the patient 530 is viewed through the HMD 510. The assessment sphere 540 may include one or more angle guidelines (e.g., lines latitude or longitude), which may be used to assess patient mobility. For example, the shoulder rotation of 0°-180° shown in FIGS. 2A-3 may be indicated by corresponding guidelines on assessment sphere 540. The assessment sphere 540 may be used to view a static patient as shown in FIG. 5A, or may be used to view a moving patient as shown in FIG. 5B. The assessment sphere 540 may be anchored to the skeletal model 550 of the patient, and may rotate or move with the patient 530. In an embodiment, the position of the assessment sphere 540 may be anchored to the top body joint of the skeletal model 550 while the rotation remains consistent with a predetermined coordinate system. For example, the assessment sphere 540 position may move with a walking patient 530, but the assessment sphere 540 rotation may be locked in an orthogonal coordinate system defined by the walls and floor of the room.

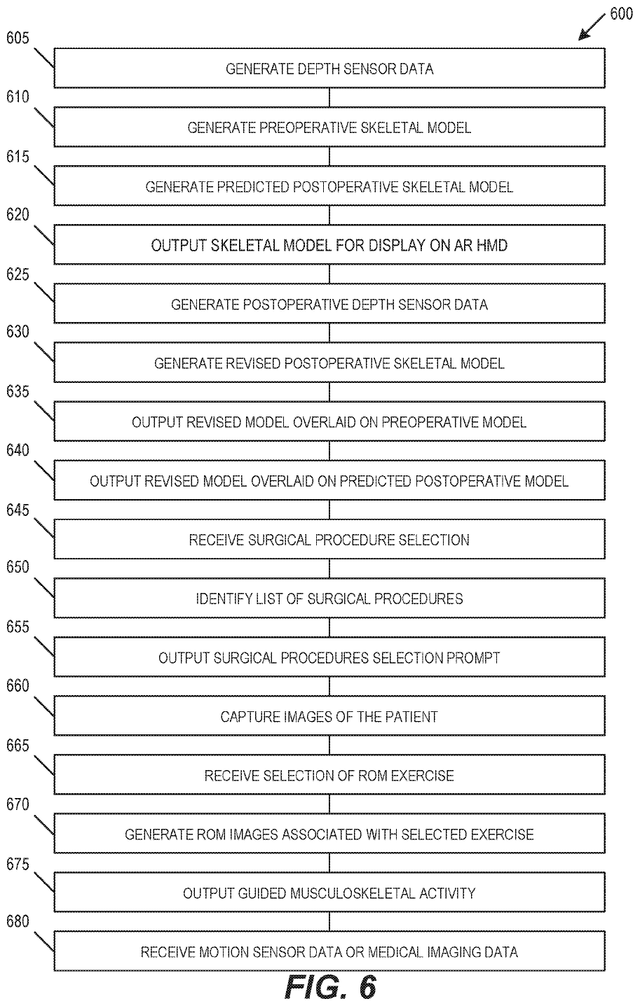

FIG. 6 illustrates a flow chart showing an AR patient assessment method 600, in accordance with some embodiments. Method 600 includes generating 605 depth sensor data for a patient during a musculoskeletal assessment activity and generating 610 a preoperative skeletal model based on the depth sensor data. Method 600 includes generating 615 a predicted postoperative skeletal model based on the preoperative skeletal model. The predicted postoperative skeletal model indicates an improved range of motion based on a surgical procedure. Method 600 includes outputting 620 the predicted postoperative skeletal model overlaid on the patient for display on an AR HMD while the patient is being viewed through the AR HMD.

In an embodiment, method 600 includes generating 625 postoperative depth sensor data for the patient during a postoperative musculoskeletal assessment activity. Method 600 may include generating 630 a revised postoperative skeletal model based on the postoperative depth sensor data. Method 600 may include outputting 635 the revised postoperative skeletal model overlaid on the preoperative skeletal model for display on the AR HMD. This may allow the viewer to contrast the revised postoperative skeletal model with the preoperative skeletal model. Method 600 may include outputting 640 the revised postoperative skeletal model overlaid on the predicted postoperative skeletal model for display on the AR HMD. This may allow the viewer to contrast the revised postoperative skeletal model with the predicted postoperative skeletal model.

In an embodiment, method 600 includes receiving 645 a surgical procedure selection, where the predicted postoperative skeletal model is further based on the surgical procedure selection. Method 600 may include identifying 650 a list of surgical procedures associated with the musculoskeletal assessment activity, and may include outputting 655 a selection prompt for the list of surgical procedures for display on the AR HMD.

In an embodiment, method 600 includes capturing 660 images of the patient, where the preoperative skeletal model is further based on the captured images of the patient. Method 600 may include receiving 665 a selection of a range of motion exercise. Method 600 may include generating 670 a plurality of range of motion images associated with the selected range of motion exercise, the plurality of range of motion images including the postoperative skeletal model overlaid on the captured images of the patient.

In an embodiment, method 600 includes outputting 675 a guided musculoskeletal activity for display on the AR HMD. The guided musculoskeletal activity may provide a patient motion instruction for conducting the musculoskeletal assessment activity.

In an embodiment, method 600 includes receiving 680 motion sensor data or medical imaging data. The motion sensor data may be received from a motion sensor attached to a patient, where the motion sensor data may characterize a musculoskeletal motion of the patient. The generation of the preoperative skeletal model may be further based on the received sensor data or on the received medical imaging data.

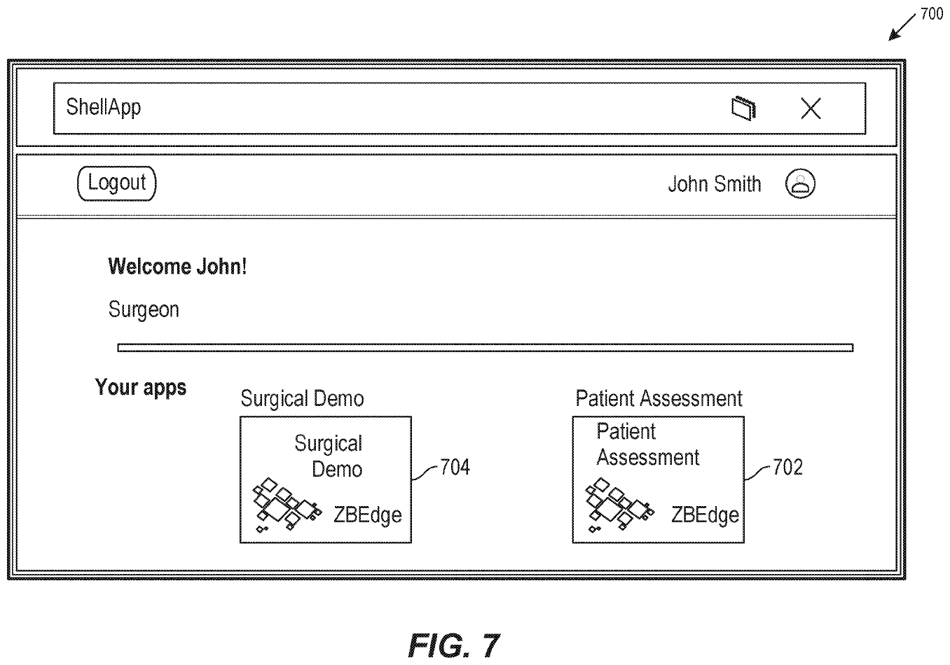

FIG. 7 illustrates a user interface 700 for selecting an application (e.g., using augmented reality), in accordance with some embodiments. The menu may be presented in virtual or augmented reality, or on a traditional screen. The user interface 700 shown in FIG. 7 is in AR, with aspects of a surrounding area visible. The AR view may be presented using glasses, a visor, goggles, or other immersion/AR setup. The menu may be selectable with a finger of a user (e.g., in real space, as detected using an AR device). A selection may be made by a clinician, a patient, or another interested party. For example, the selection may include a patient assessment in AR with indication 702, or a surgical demonstration in AR with indicator 704.

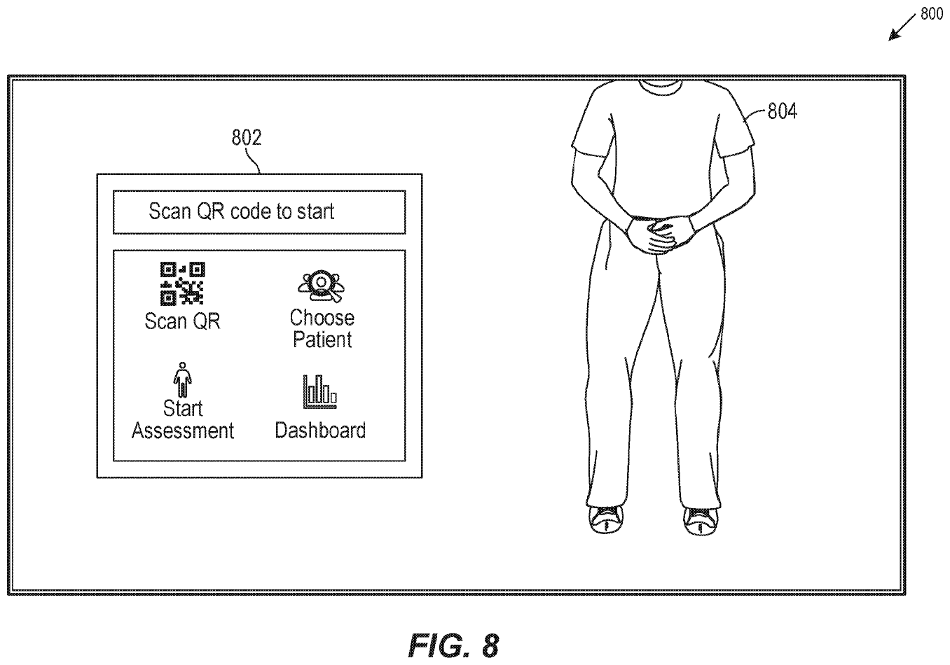

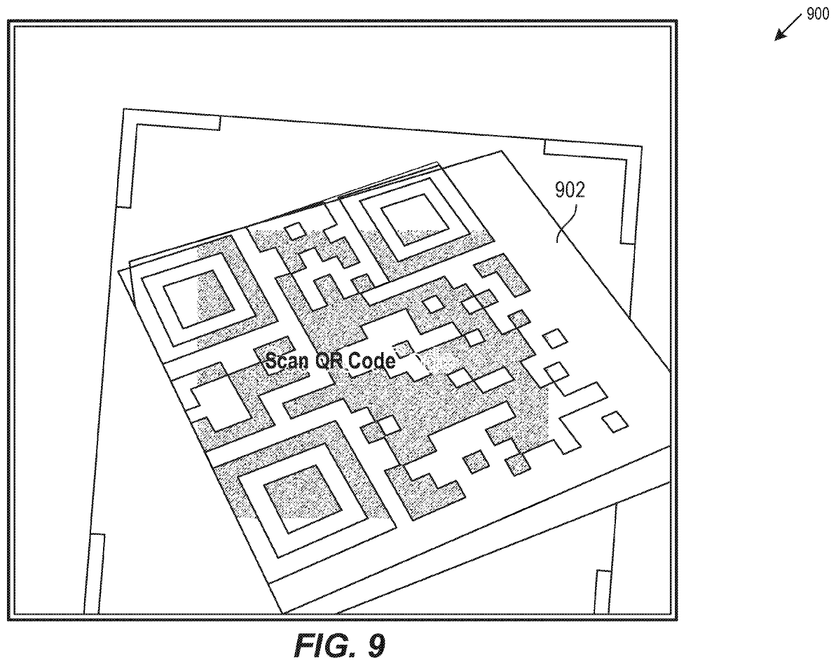

FIG. 8 illustrates a first user interface 800 for patient selection, in accordance with some embodiments. FIG. 8 includes a menu 802 (e.g., an AR menu) displaying a selectable indicator with a representation of a QR code. Upon selection of the selectable indicator, a camera view may be presented (e.g., as shown in AR with reticles framing a capture area in FIG. 9). Other selectable options in menu 802 include choosing a patient, starting an assessment, or viewing a dashboard. In an example where the menu 802 is shown in AR, the menu 802 may be automatically aligned, such as to a side of a recognized patient, on a wall, etc.

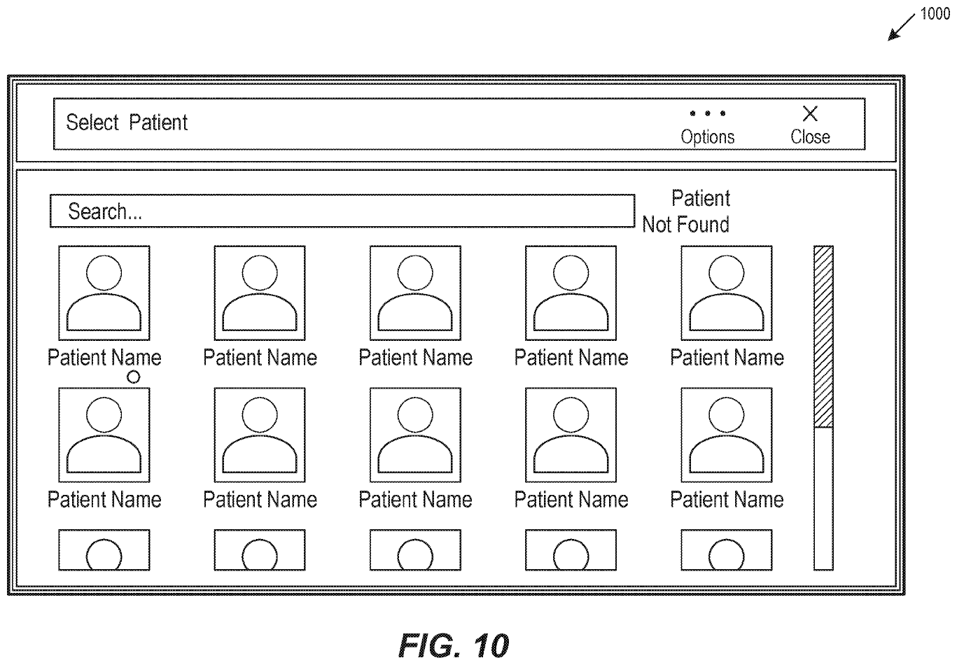

FIG. 9 illustrates a second user interface 900 for patient selection, in accordance with some embodiments. A QR code 902 may be scanned using a camera (e.g., attached to an AR device displaying the menu 802. The QR code 902 may be patient-specific, clinician-specific, assessment-specific, instrument-specific, a combination, or the like. The QR code 902 may be scanned as shown in FIG. 9, to cause a new menu to be displayed. After scanning the QR code 902, an AR display (e.g., a menu) may be automatically populated with information, such as patient information, a list of a surgeon's patients, instrument information, assessment information, a list of facility or group patients, etc. An example list of patients is shown in FIG. 10, discussed below.

In an example, the QR code may server the purpose of finding a location of the camera, which may include a lidar camera. The camera may provide the positioning of the spatial location of the skeletal joints which is in camera coordinates. The AR device may convert those joint coordinates to real world coordinates.

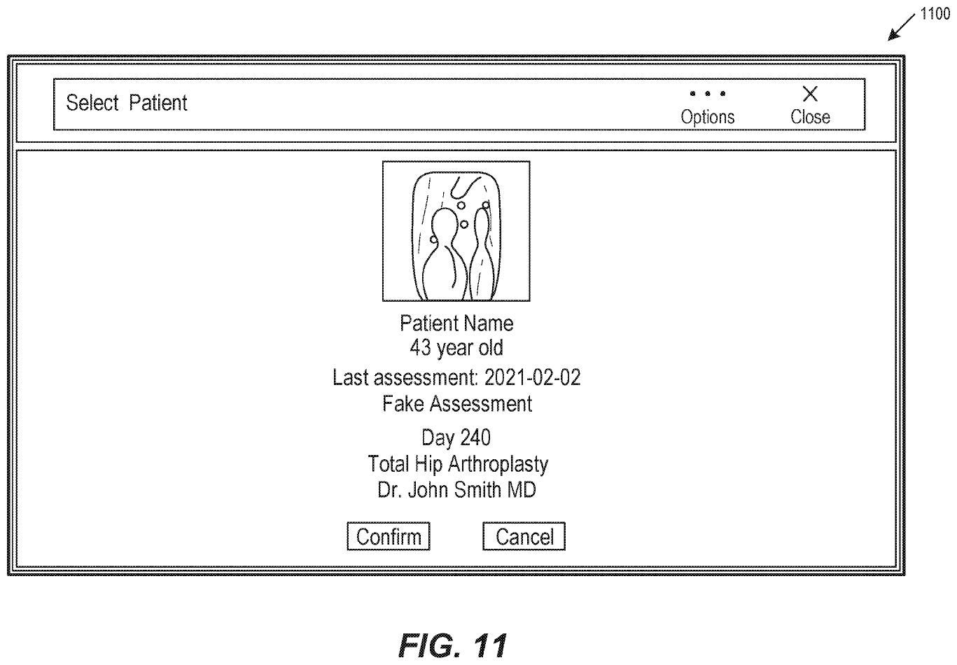

FIG. 10 illustrates a user interface 1000 for selecting a patient, in accordance with some embodiments. The user interface 1000 may be displayed in response to a user selection, scanning a QR code (e.g., as described above with respect to FIGS. 8-9), a user logging in, or the like. The user interface 1000 may be moved within a visible field when presented in AR. The user interface 1000 may be fixed to different locations in the real world, such as a wall, or it may move by maintaining a fixed distance to a user wearing an AR device. In an example, a patient may be selected from the user interface 1000. When selected, further details of the patient may be displayed (e.g., as discussed below with respect to FIG. 11).

FIG. 11 illustrates a user interface 1100 for displaying patient information, in accordance with some embodiments. The user interface 1100 may be displayed in response to a patient selection (e.g., on user interface 1000, a vocal command, a gesture, etc.). When the patient is selected, details for the patient may be shown in user interface 1100, such as name, age, last assessment, procedure (e.g., done or to be done), surgeon name, etc. The user of the AR device displaying the user interface 1100 may confirm or cancel the patient selection. In some examples, the patient may not be found, such as when a QR code is not available, or when the patient is a new patient and needs a new entry. In these examples, a virtual keyboard may be displayed for manual entry of a patient's name or other details. After selection to confirm the patient, an assessment user interface may be displayed.

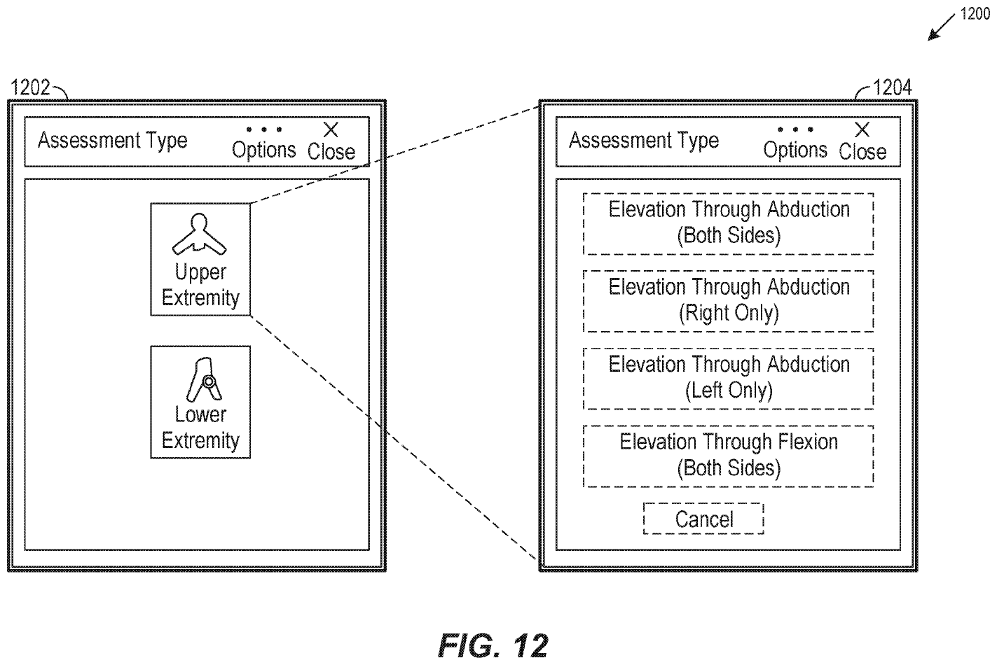

FIG. 12 illustrates assessment user interface 1202 for selecting an assessment, in accordance with some embodiments. The assessment user interface 1202 identifies broad selectable assessment types, such as upper extremity or lower extremity. After selection of the broad category, the assessment user interface 1204 may be displayed with different categories or sub-categories of assessments for selection. The displayed options may be limited to those applicable to a previously selected patient, or a larger set of assessment types may be displayed. The displayed assessment types in assessment user interface 1204 include may include multiple parts (e.g., a first assessment corresponding to a first leg or first movement and a second assessment corresponding to a second leg or second movement). Example assessments may include those shown in Tables 1 and 2 below, such as single joint, single plane movements for Upper Extremity or multi plane, multi joint movements for Lower Extremity. These two example assessments may be referred to categorically as single joint, single plane traditional active range of motion assessments or multi joint, multi plane functional assessments.

TABLE 1
Upper Extremity Assessments

Elevation Through Abduction (Both Sides)
Elevation Through Abduction (Right Only)
Elevation Through Abduction (Left Only)
Elevation Through Flexion (Both Sides)
Elevation Through Flexion (Right Only)
Elevation Through Flexion (Left Only)
Extension (Both Sides)
Extension (Right Only)
Extension (Left Only)
Horizontal Adduction (Both Sides)
Horizontal Adduction (Right Only)
Horizontal Adduction (Left Only)
Internal Rotation (Both Sides)
Internal Rotation (Right Only)
Internal Rotation (Left Only)
External Rotation (Both Sides)
External Rotation (Right Only)
External Rotation (Left Only)

TABLE 2
Lower Extremity Assessments

Sit to Stand
Squat
Single Leg Balance (Left)
Single Leg Balance (Right)
Gait

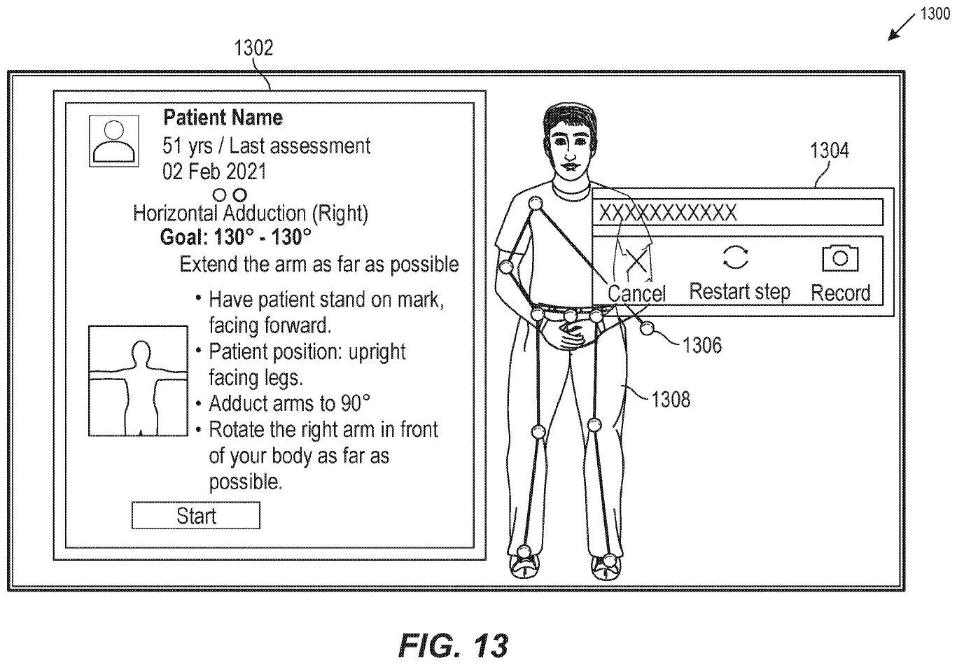

After selection of the particular assessment, an AR assessment may be displayed, for example as discussed below with respect to FIG. 13.

FIG. 13 illustrates user interfaces 1300 for displaying aspects of an AR assessment, in accordance with some embodiments. FIG. 13 illustrates a first user interface 1302, a second user interface 1304, and a skeletal overlay 1306 on a patient 1308 for displaying aspects of an AR assessment. The interfaces or components of FIG. 13 may be displayed in response to receiving a selection of an assessment (e.g., on assessment user interface 1204 of FIG. 12, for example). A menu 1304 may be displayed including a name of the assessment, with selectable options related to the assessment to start, to record (e.g., from the AR display), to cancel, to restart a step, or the like. A second menu 1302 may be displayed including patient demographic information, instructions for the patient to complete the exercise (which may include instructions for the patient, or instructions for the clinician to use to instruct the patient, e.g., which may by written differently, such as including lay terminology or clinical terminology), or the like. The second menu 1302 may include a selectable indicator to start the assessment. The second menu 1302 may include details related to a goal of the assessment (e.g., a goal range of motion, which may be general or specific to the patient, such as postoperatively based on a completed surgical procedure, physical therapy, time since the procedure, etc.). The second menu 1302 may include a component to display a virtual demonstration of the assessment. The component may be activated automatically or by a user of the AR device.

The menus 1302 and 1304 may be separately moveable, may be fixed to a portion of a room, may be relatively fixed to the patient 1308 or other moving object, may be fixed to each other, may be set at a fixed distance away from a wearer of an AR display presenting the menus 1302 or 1304, or the like.

In an example, a skeletal frame 1306 may be displayed overlaid on the patient 1308. The skeletal frame 1306 may include joints, segments (e.g., corresponding to bones or other body parts), or the like. The skeletal overlay may move with the patient 1308, with the augmented image of the skeletal overlay tracking real world movements of the patient 1308. While the skeletal frame 1306 is described as tracking real world movements of the patient 1308, the skeletal frame 1306 may also be displayed in a manner that appears to move relative to a wearer of an AR device presenting the skeletal frame 1306. For example, when the wearer moves, the perspective of the skeletal frame 1306 may change such that it remains between the wearer and the patient 1308. In other examples, the skeletal frame 1306 may not move relative to the wearer, such that the skeletal frame 1306 may become partially or fully obscured if the field of vision of the wearer changes.

The skeletal frame 1306 may be generated from the patient 1308, for example using a camera, such as a lidar camera, a depth camera, or the like. The camera may be part of the AR device or separate. Data from the camera may be sent, for example via an API, to a processor executing control over display of the AR device, and the skeletal frame 1306 may be output for display using the AR device based on the received data.

In an example, a lidar camera may be used to capture and identify the patient 1308 via projected light. The skeletal frame 1308 may be derived from the lidar camera data, for example using image recognition and a skeletal assignment, which may be optionally personalized to the patient 1306. The visualization of the skeletal frame 1308 may be rendered and displayed via an AR device. Range of motion data for the patient 1306 may be determined based on movement of the patient 1306 (e.g., as captured via the lidar camera, a camera on the AR device, etc.), and compared to expected movement in the space based on known kinematics of skeletal frames, and the patient's 1306 captured or known anatomy (e.g., height). Information for the patient 1306 (e.g., height, arm width, etc.) may be stored in a connected health cloud.

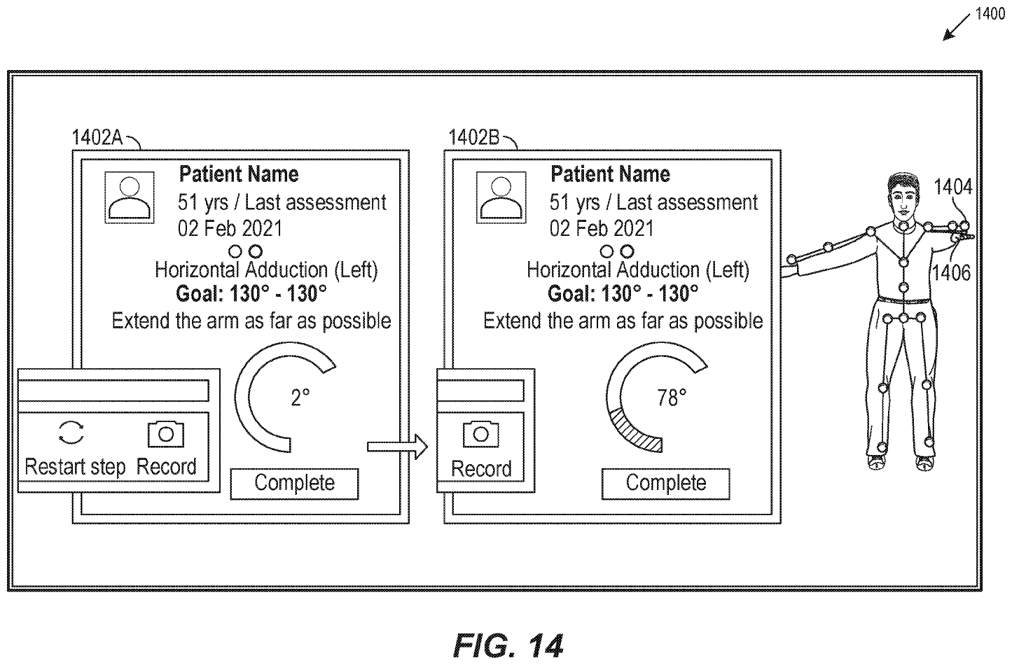

After receiving a selection to start (e.g., on one of the menus 1302 or 1304, via gesture, audio command, etc.), a real-time indicator of range of motion may be displayed, such as described below in proceed with respect to FIG. 14.

FIG. 14 illustrates user interfaces 1400 for displaying aspects of an AR assessment and skeletal overlay, in accordance with some embodiments. FIG. 14 illustrates a user interface (e.g., 1402 and 1404) and component (e.g., with portions 1406 and 1408) for displaying aspects of an AR assessment and skeletal overlay. User interface 1402 includes a real-time indicator of range of motion of a patient. During an assessment, as the patient moves, the real-time indicator may update with a present or total range of motion achieved (e.g., displayed as 2 degrees in user interface 1402, such as when the assessment has just started). A “start” selectable indicator of the user interface 1402 may be changed to a “complete” or “finish” selectable indicator (e.g., after progress has been made and the range of motion has changed), which, when selected, may stop the assessment.

An indication in user interface 1404 of the range of motion progress (e.g., towards a goal range of motion), such as using a completion bar, circle, etc. In some examples, effects may be added or changed in the user interface 1404 to indicate progress, such as a color change, a popup, a sound, or other display, for example to indicate an amount or degree of progress. A degree of progress may correspond to passing a previous personal record, achieving a range of motion goal, or the like.

User interface 1404 includes a skeletal overlay on a user, which includes portions 1406 and 1408. A display enhancement may be shown with the skeletal overlay to indicate a path of motion (e.g., a goal at portion 1408 and a current portion 1406 of an extremity), a target or goal, a starting point, or the like. The display enhancement may be shown in real-time and modified as the patient moves during the assessment.

User interfaces 1402 and 1404 illustrate both user interface menus and the skeletal frame of a patient. The joint display of the user interface menus and the skeletal frame allows a user (e.g., a clinician, such as a surgeon) to view the patient, the skeletal frame, and data related to the movement all within one view. This improves the visibility of information by allowing the clinician to not need to look at a screen (e.g., losing sight of the patient). The skeletal frame may provide depth information in some examples. The clinician or other user of the AR device may move, and the user interface components may move with the clinician or remain static (e.g., near the patient). In some examples, the user interface components may appear to rotate in space to the user of the AR device to allow the user to view the user interface components at any angle, while also allowing the user to gain different perspectives of the patient and the skeletal frame. This allows the user to view accurate cardinal plane motion, which may be viewed both visually on the skeletal frame and optionally in a user interface component as a value.

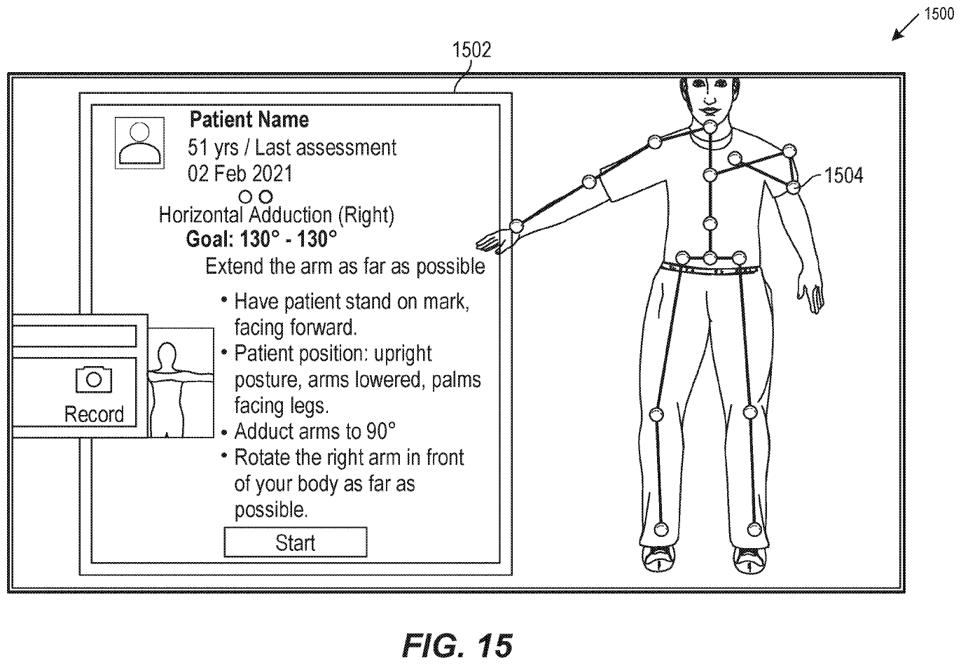

In an example, after the assessment is completed, the range of motion (e.g., full range or quality of motion) has been achieved, or after a completion indication is selected, an AR assessment may be displayed, as described below with respect to FIG. 15.

FIG. 15 illustrates a user interfaces 1500 for displaying aspects of an AR assessment and skeletal overlay, in accordance with some embodiments. FIG. 15 illustrates a user interface 1502 and component 1504 for displaying aspects of an AR assessment and skeletal overlay. The user interface 1502 includes details of the assessment (e.g., completed as described above). A skeletal overlay or a display enhancement may be retained for viewing. The user interface 1502 may include an indication of a maximum achieved range of motion, displayed for example relative to a goal or personal best. The component 1504 may show an end location for an extremity during the range of motion assessment, for example to show the user the progress or achievement.

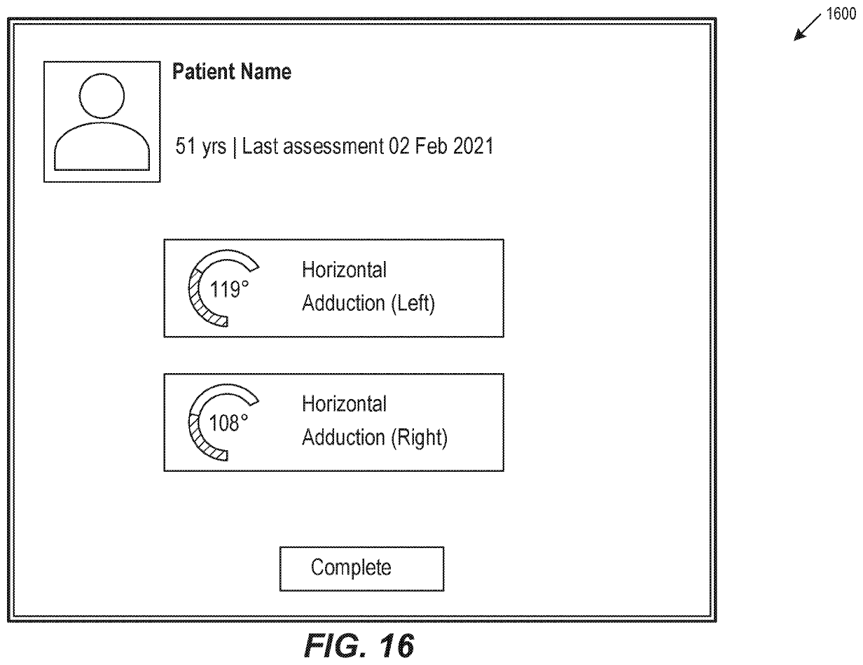

When an assessment has multiple parts (e.g., two limbs, two exercises, etc.), the system may move on to a next part after selection of a “complete” indication (e.g., as described above). In some examples, the next part may start right away, while in other examples, the user may select a “start” indication to start the next part. When one or all parts of an assessment are completed, total results of the AR assessment may be displayed, as described below with respect to FIG. 16.

FIG. 16 illustrates a user interface 1600 for displaying results of an AR assessment, in accordance with some embodiments. The user interface 1600 may include a menu indicating results of the assessment. In the example shown in FIG. 16, range of motion degrees (e.g., 119 and 108) are displayed for a horizontal adduction assessment for both a left and right extremity.

When a user selects the “complete” indication on the user interface 1600 indicating that the user is done reviewing the AR assessment results, the system may return to a previous menu for further assessment, if needed, or for completion, storage, or sending of the assessment results. The system may provide further instruction (e.g., exercises to work on improving range of motion, education about benefits of improving range of motion, instructions to contact a clinician, etc.).

An augmented or virtual view of patient anatomy may be displayed after the AR assessment is complete, in some examples. The patient anatomy may be displayed according to a role of the person viewing the anatomy, such as a patient view or a clinician view. A patient view may be more simplistic than a clinician view in terms of anatomy or clinical information. In some examples, a patient view may include additional information, such as explanations, education materials, color or other display effects, or the like with the patient anatomy.

A completed or yet to be completed procedure may be shown (e.g., a roadmap with an indication of where a patient is along the roadmap). Augmented patient anatomy may be displayed in various states, such as anatomy before, during, or after the procedure, preoperatively with predictive viewing of an outcome, postoperatively, such as to compare to a preoperative predicted outcome, or the like. In an example, patient anatomy may be shown in motion, statically, in an exploded view, or the like. Patient anatomy displayed in the augmented reality view may be rotatable, moveable, enlargeable or shrinkable, etc.

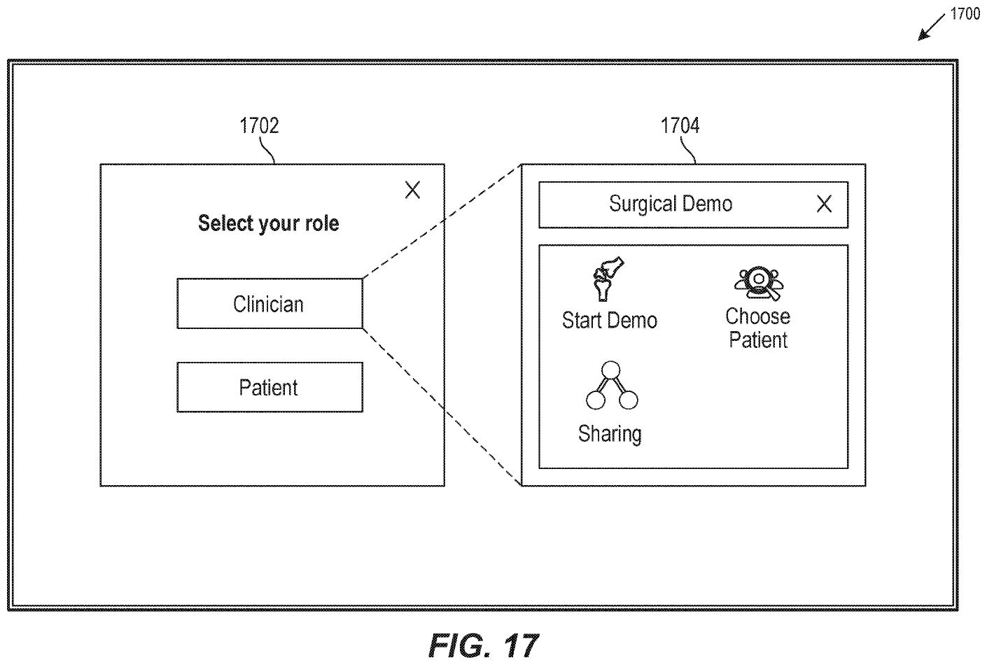

FIG. 17 illustrates user interfaces 1700 for selecting a surgical demonstration in augmented reality, in accordance with some embodiments. A user interface 1702 allows a user to select a role, such as clinician or patient. A user interface 1704 may be displayed after a selection is made on user interface 1702. User interface 1704 may include selections for starting a demo, choosing a patient (e.g., when the user is a clinician), sharing, or the like. In an example, when the user selects “patient” in the user interface 1702, the user interface 1704 may be skipped, for example going to a patient select screen for example to confirm the patient.

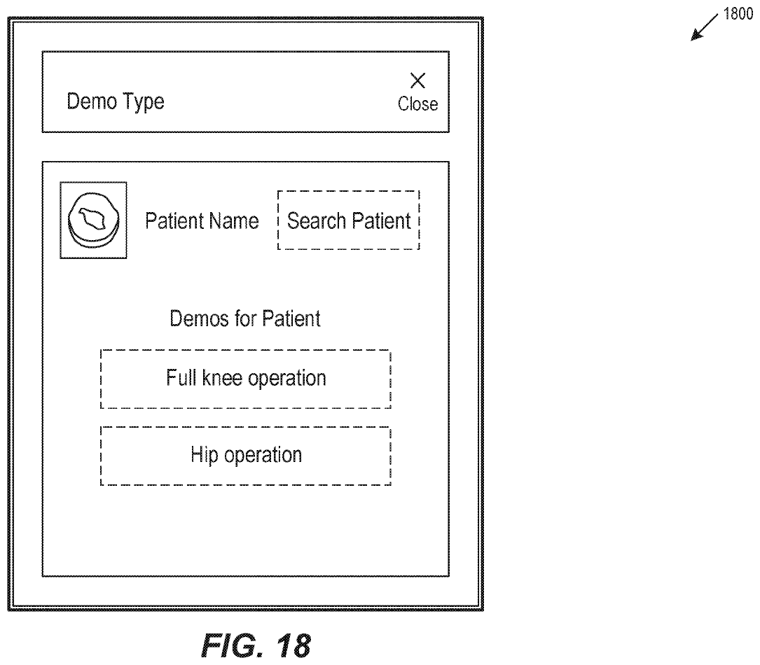

FIG. 18 illustrates a user interface 1800 for selecting a surgical demonstration in augmented reality. When a user selects an indication for starting a demo (e.g., on user interface 1104 or FIG. 11B), the user interface 1800 may display viewable demos. The user may select a demo for viewing (e.g., corresponding to a patient, a clinician, a type of procedure, etc.). The user interface 1800 includes demos for full knee operation or hip operation. A patient may be selected to personalize the demo in some examples. The demo may include an AR demonstration as described below, which may correspond to an upcoming or previously completed surgical procedure.

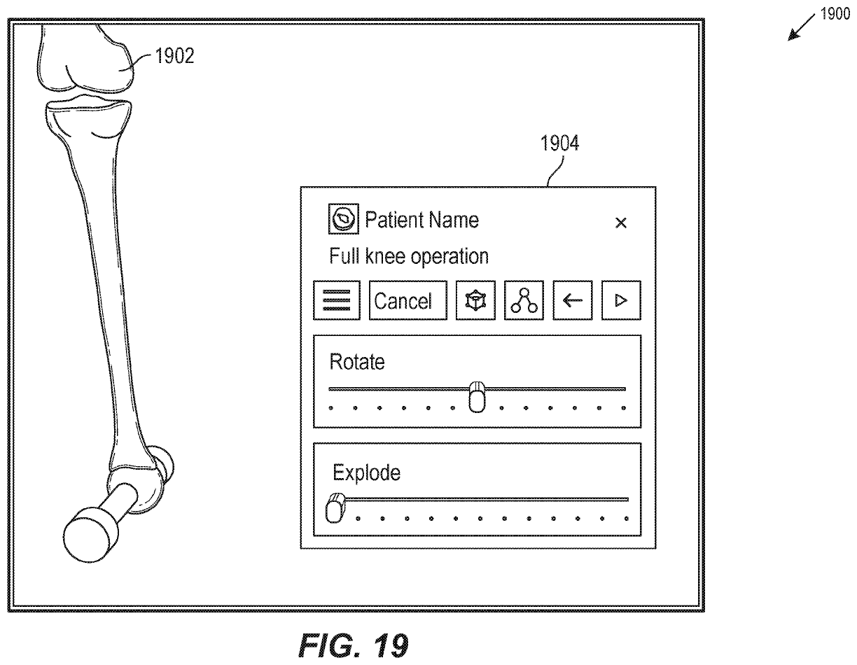

FIG. 19 illustrates user interfaces 1900 for displaying aspects of an AR demonstration and 3D bone model, in accordance with some embodiments. FIG. 19 illustrates a user interface 1904 and component 1902 for displaying aspects of an AR demonstration and 3D bone model. The component 1902 may include a virtually manipulable 3D display of the 3D bone model, presented in AR. The 3D bone model may include a custom 3D model based on patient imaging in some examples. The control user interface 1904 may display components such as rotate, explode, play, cancel, share, etc. for controlling the display of the component 1902. In some examples, the component 1902 may be controlled by “moving” the component 1902 (e.g., using a hand or gesture).

In an example, selecting a “play” button on the user interface 1904 causes full rotation or range of motion of the 3D bone model to be shown. When displaying the 3D bone model in AR, resections, installation of trials, exploded views, implants, rotation, or the like may be displayed (e.g., as an animation). In this way, a user may view an end-to-end display in 3D AR of the procedure. A clinician user may use the 3D AR display to visualize or issue spot, while a patient user may be given a visual walkthrough of the procedure. When viewing is complete, a user may select a “confirm” or “check” button.

The component 1902 illustrates a three-dimensional rendering of patient anatomy, an implant, a trial, etc. in an augmented reality display in accordance with some examples. The AR display includes the component 1902, which may include anatomy of a patient, for example generated using an x-ray, an MRI, a CT scan, or the like. The AR display may illustrate an animation, or allow control of or movement of the three-dimensional virtual representation of the patient anatomy or an implant in the component 1902 (e.g., a bone). The three-dimensional virtual representation may be interacted with by a clinician viewing the AR display, for example using a button, a remote, a gesture, an input on a menu of the user interface 1904, etc. The interaction may manipulate the component 1902, for example rotate, move, zoom, etc., the component 1902. By manipulating the component 1902, the clinician may visualize a surgical procedure, such as pre-operatively or post-operatively.

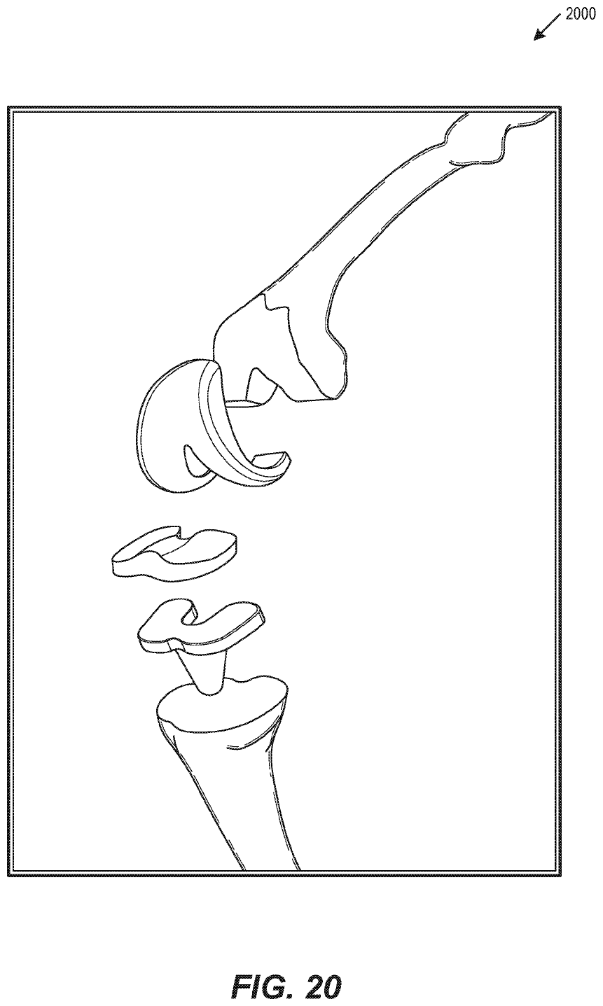

FIG. 20 illustrates an example 3D bone model AR view 2000, in accordance with some embodiments. The AR view 2000 includes an exploded view of a 3D bone model, which may include an implant or trial, visual indications of resections, etc. The AR view 2000 may be controlled, for example to expand or compress (e.g., reassemble), to rotate, move, etc. In some examples, a component may be selected, such as a trial or implant, which may then be removed, moved independently of the model, swapped out for another, changed in size, or the like. This allows a clinician to view an augmented model and quickly visualize different sized trials or implants. In some examples resections may be modified, for example depth of cut, angle of cut, etc. to allow a clinician to visualize resection changes.

In an example, the AR view 2000 includes a demonstration system of for a surgical procedure. In some examples, the AR view 2000 may use non-patient specific bone anatomy, while in other examples, patient-specific bone anatomy (e.g., based on imaging of the patient) may be used. The AR view 2000 may be used to show medical device components, such as a knee system (e.g., total or partial), a hip system, etc.

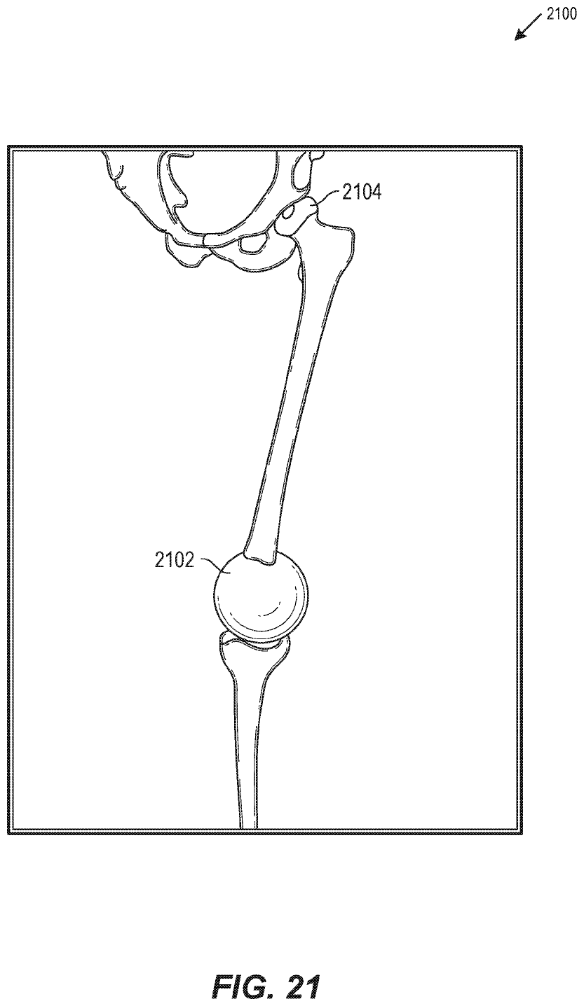

FIG. 21 illustrates an example 3D bone model in an AR view 2100, in accordance with some embodiments. The AR view 2100 includes a visual indicator 2102 of where movement of the model may occur. In the example AR view 2100, the 3D bone model may be stable (e.g., unmovable) except for the movement location indicated by the visual indicator 2102 (here, a blue sphere). A second visual indicator 2104 may be used to indicate a rotation joint that anchors the movement performed by moving the visual indicator 2102. For example, in AR view 2100, the visual indicator 2102 is at a knee joint, and when moved, causes the femur to rotate at the hip joint, as shown by the second visual indicator 2104. Visual indicators may be displayed uniquely (e.g., with a sphere, with a different color, changing when a cursor or finger hovers over them, etc.). In AR view 2100, the knee is moveable to show hip rotation or range of motion, for example of a patient-specific 3D bone model.

AR views 2000 or 2100 may be used to display user interface components, models, techniques, skeletal frames, or the like as described herein, for example in a multi-user system. In some examples, the multi-user system may be used by a clinician and a patient with synced or connected AR devices. The clinician's AR device may be used to control or demonstrate aspects of patient recovery, surgical procedures, or the like, in the patient's AR device. In some examples, the anatomy shown in AR views 2000 or 2100 may move in a pre-defined way (e.g., animated). In other examples, a clinician may control the anatomy by rotating, spinning, exploding, playing, pausing, speeding up, slowing down, or the like.

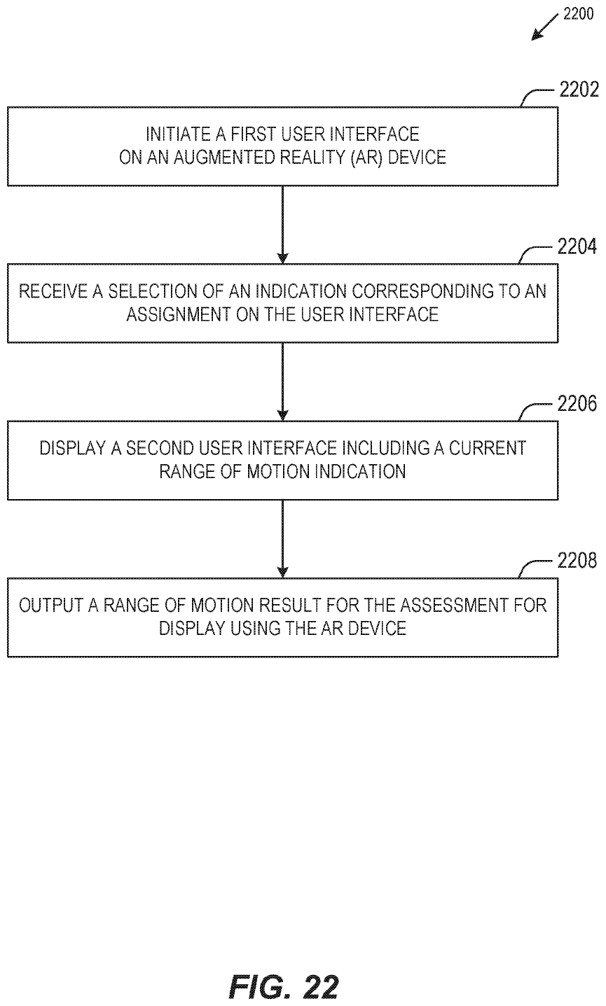

FIG. 22 illustrates a flowchart showing a technique 2200 for performing a patient assessment using augmented reality in accordance with at least one example of this disclosure. The technique 2200 includes an operation 2202 to initiate, using an augmented reality (AR) device, a first user interface including selectable indications corresponding to assessments. In an example a patient may be selected using an AR user interface (or multiple AR user interfaces).

Technique 2200 includes an operation 2204 to receive a selection of a selectable indication on the first user interface, the selectable indication corresponding to an assignment on the user interface. The technique 2200 includes an operation 2206 to display a second user interface including a current range of motion indication corresponding to a current position of a patient performing the assessment, the patient visible via the AR device. The technique 2200 includes an operation 2208 to output a range of motion result for the assessment for display using the AR device. In an example, the technique 2200 may include displaying a skeletal overlay on a patient, instead of or in addition to operations 2206 or 2208.

The skeletal overlay may be displayed with a user interface in an AR display via the AR device. The skeletal overlay may move as the patient moves. The user interface may be controlled, by the user or automatically, to follow a view of the user of the AR display, or may remain static or fixed to a particular distance from an object (e.g., the patient). When the user of the AR device moves, the user interface may follow or may rotate (e.g., when set to be a fixed distance), etc.

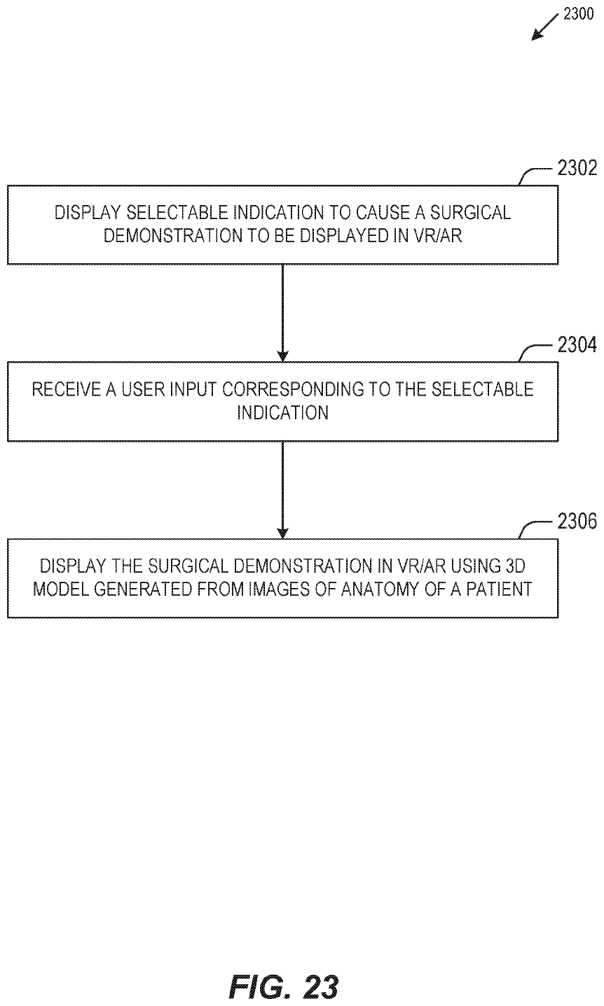

FIG. 23 illustrates a flowchart showing a technique 2300 for displaying a surgical demonstration using augmented reality in accordance with at least one example of this disclosure. The technique 2300 includes an operation 2302 to display a user interface including a selectable indication, which when selected, causes a surgical demonstration to be displayed in virtual or augmented reality using a virtual or augmented reality display device. The technique 2300 includes an operation 2304 to receive a user input corresponding to the selectable indication. The technique 2300 includes an operation 2306 to display the surgical demonstration in virtual or augmented reality using a 3D model generated from images of anatomy of a patient, the surgical demonstration including at least one 3D display of: a full rotation of the 3D model, a range of motion of the 3D model, a resection of the 3D model, an installation of a trial or implant in the 3D model, or an exploded view of the 3D model.

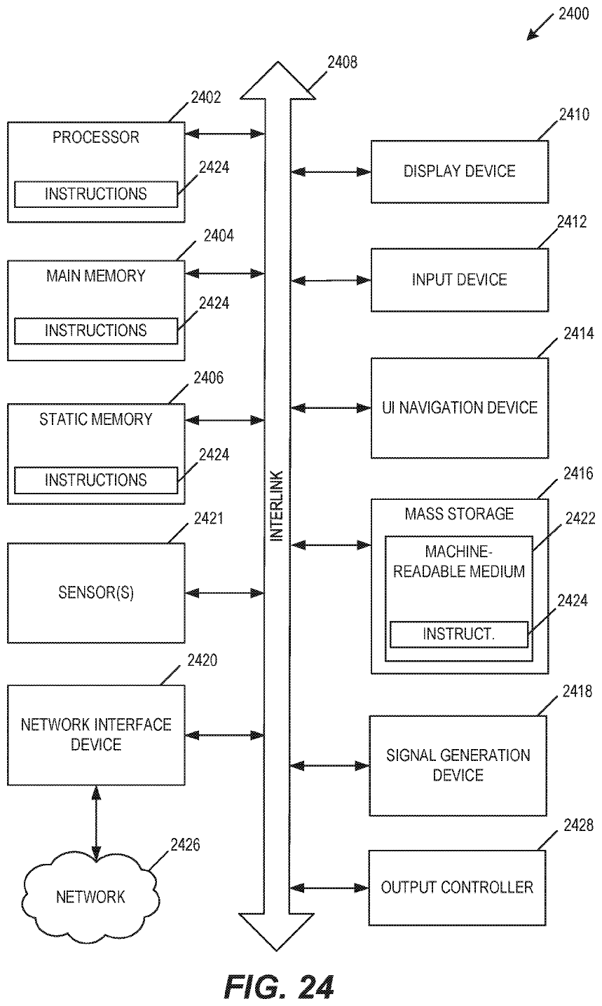

FIG. 24 illustrates an example of a block diagram of a machine 2400 upon which any one or more of the techniques (e.g., methodologies) discussed herein may perform in accordance with some embodiments. In alternative embodiments, the machine 2400 may operate as a standalone device or may be connected (e.g., networked) to other machines. In a networked deployment, the machine 2400 may operate in the capacity of a server machine, a client machine, or both in server-client network environments. The machine 2400 may be a personal computer (PC), a tablet PC, a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (Saas), other computer cluster configurations.

Examples, as described herein, may include, or may operate on, logic or a number of components, modules, or like mechanisms. Such mechanisms are tangible entities (e.g., hardware) capable of performing specified operations when operating. In an example, the hardware may be specifically configured to carry out a specific operation (e.g., hardwired). In an example, the hardware may include configurable execution units (e.g., transistors, circuits, etc.) and instructions contained on a computer readable medium, where the instructions configure the execution units to carry out a specific operation when in operation. The configuring may occur under the direction of the execution units or a loading mechanism. Accordingly, the execution units are communicatively coupled to the computer readable medium when the device is operating. For example, under operation, the execution units may be configured by a first set of instructions to implement a first set of features at one point in time and reconfigured by a second set of instructions to implement a second set of features.

Machine (e.g., computer system) 2400 may include a hardware processor 2402 (e.g., a central processing unit (CPU), a graphics processing unit (GPU), a hardware processor core, or any combination thereof), a main memory 2404 and a static memory 2406, some or all of which may communicate with each other via an interlink (e.g., bus) 2408. The machine 2400 may further include a display unit 2410, an alphanumeric input device 2412 (e.g., a keyboard), and a user interface (UI) navigation device 2414 (e.g., a mouse). In an example, the display unit 2410, alphanumeric input device 2412 and UI navigation device 2414 may be a touch screen display. The display unit 2410 may include goggles, glasses, an augmented reality (AR) display, a virtual reality (VR) display, or another display component. For example, the display unit may be worn on a head of a user and may provide a heads-up-display to the user. The alphanumeric input device 2412 may include a virtual keyboard (e.g., a keyboard displayed virtually in a VR or AR setting.

The machine 2400 may additionally include a storage device (e.g., drive unit) 2416, a signal generation device 2418 (e.g., a speaker), a network interface device 2420, and one or more sensors 2421, such as a global positioning system (GPS) sensor, compass, accelerometer, or another sensor. The machine 2400 may include an output controller 2428, such as a serial (e.g., universal serial bus (USB), parallel, or other wired or wireless (e.g., infrared (IR), near field communication (NFC), etc.) connection to communicate or control one or more peripheral devices.

The storage device 2416 may include a machine readable medium 2422 that is non-transitory on which is stored one or more sets of data structures or instructions 2424 (e.g., software) embodying or utilized by any one or more of the techniques or functions described herein. The instructions 2424 may also reside, completely or at least partially, within the main memory 2404, within static memory 2406, or within the hardware processor 2402 during execution thereof by the machine 2400. In an example, one or any combination of the hardware processor 2402, the main memory 2404, the static memory 2406, or the storage device 2416 may constitute machine readable media.

While the machine readable medium 2422 is illustrated as a single medium, the term “machine readable medium” may include a single medium or multiple media (e.g., a centralized or distributed database, or associated caches and servers) configured to store the one or more instructions 2424.

The term “machine readable medium” may include any medium that is capable of storing, encoding, or carrying instructions for execution by the machine 2400 and that cause the machine 2400 to perform any one or more of the techniques of the present disclosure, or that is capable of storing, encoding, or carrying data structures used by or associated with such instructions. Non-limiting machine-readable medium examples may include solid-state memories, and optical and magnetic media. Specific examples of machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 2424 may further be transmitted or received over a communications network 2426 using a transmission medium via the network interface device 2420 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, as the personal area network family of standards known as Bluetooth® that are promulgated by the Bluetooth Special Interest Group, peer-to-peer (P2P) networks, among others. In an example, the network interface device 2420 may include one or more physical jacks (e.g., Ethernet, coaxial, or phone jacks) or one or more antennas to connect to the communications network 2426. In an example, the network interface device 2420 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 2400, and includes digital or analog communications signals or other intangible medium to facilitate communication of such software.

Each of these non-limiting examples may stand on its own, or may be combined in various permutations or combinations with one or more of the other examples.

Example 1 is a system for augmented reality patient assessment, the system comprising: an augmented reality (AR) head-mounted display (HMD); a depth sensor to generate depth sensor data for a patient during a musculoskeletal assessment activity; processing circuitry; and a memory that includes instructions, the instructions, when executed by the processing circuitry, cause the processing circuitry to: generate a skeletal model based on the depth sensor data; track a patient motion during the musculoskeletal assessment activity; determine a current ROM based on the patient motion; and output the current ROM overlaid on the patient for display on the AR HMD while the patient is being viewed through the AR HMD.

In Example 2, the subject matter of Example 1 includes instructions further causing the processing circuitry to: receive a selection of the musculoskeletal assessment activity; and output a description of the musculoskeletal assessment activity for display on the AR HMD while the patient is being viewed through the AR HMD.

In Example 3, the subject matter of Examples 1-2 includes instructions further causing the processing circuitry to: determine a target ROM based on the musculoskeletal assessment activity; and output a graphical indication of the target ROM for display on the AR HMD while the patient is being viewed through the AR HMD.

In Example 4, the subject matter of Examples 1-3 includes instructions further causing the processing circuitry to output a guided musculoskeletal activity for display on the AR HMD, the guided musculoskeletal activity providing a patient motion instruction for conducting the musculoskeletal assessment activity.

In Example 5, the subject matter of Examples 1˜4 includes instructions further causing the processing circuitry to receive motion sensor data from a motion sensor attached to a patient, the motion sensor data characterizing a musculoskeletal motion of the patient; wherein the generation of the skeletal model is further based on the sensor data.

In Example 6, the subject matter of Examples 1-5 includes instructions further causing the processing circuitry to receive medical imaging data of a musculoskeletal joint of the patient; wherein the generation of the skeletal model is further based on the medical imaging data.

In Example 7, the subject matter of Examples 1-6 includes instructions further causing the processing circuitry to: receive a selection of a model surgical procedure; generate a patient procedure model based on the model surgical procedure and the skeletal model; and output the patient procedure model for display on the AR HMD while the patient is being viewed through the AR HMD.

In Example 8, the subject matter of Examples 1-7 includes instructions further causing the processing circuitry to: generate a predicted postoperative skeletal model based on the skeletal model, the skeletal model including a preoperative skeletal model, the predicted postoperative skeletal model including an improved range of motion (ROM) based on a surgical procedure; and output the predicted postoperative skeletal model overlaid on the patient for display on the AR HMD while the patient is being viewed through the AR HMD.

In Example 9, the subject matter of Example 8 includes the depth sensor further to generate postoperative depth sensor data for the patient during a postoperative musculoskeletal assessment activity; and the instructions further causing the processing circuitry to: generate a revised postoperative skeletal model based on the postoperative depth sensor data; and output the revised postoperative skeletal model overlaid on the preoperative skeletal model for display on the AR HMD.

In Example 10, the subject matter of Example 9 includes instructions further causing the processing circuitry to output the revised postoperative skeletal model overlaid on the predicted postoperative skeletal model for display on the AR HMD.

In Example 11, the subject matter of Examples 8-10 includes instructions further causing the processing circuitry to receive a surgical procedure selection, wherein the predicted postoperative skeletal model is further based on the surgical procedure selection.

In Example 12, the subject matter of Example 11 includes instructions further causing the processing circuitry to: identify a list of surgical procedures associated with the musculoskeletal assessment activity; and output a selection prompt for the list of surgical procedures for display on the AR HMD.

In Example 13, the subject matter of Examples 8-12 includes an image sensor to capture a plurality of images of the patient, wherein the preoperative skeletal model is further based on the plurality of images of the patient.

In Example 14, the subject matter of Example 13 includes instructions further causing the processing circuitry to: receive a selection of a ROM exercise; and generate a plurality of ROM images associated with the ROM exercise, the plurality of ROM images including the predicted postoperative skeletal model overlaid on the plurality of images of the patient.

In Example 15, the subject matter of Examples 1-14 includes a patient AR HMD, the instructions further causing the processing circuitry to: output the predicted skeletal model for display on the AR HMD while the patient is being viewed by a medical practitioner through the AR HMD; capture an image of the patient as viewed by the medical practitioner through the AR HMD; and output the predicted skeletal model overlaid on the image of the patient for display on the patient AR HMD.

In Example 16, the subject matter of Examples 1-15 includes instructions further causing the processing circuitry to output a multiple pose skeletal model, the multiple pose skeletal model configured to display a plurality of positions of a patient body part based on the improved ROM when the multiple pose skeletal model is overlaid on the patient for display on the AR HMD while the patient is being viewed through the AR HMD.

In Example 17, the subject matter of Example 16 includes instructions further causing the processing circuitry to output a motion skeletal model overlaid on the patient for display on the AR HMD while the patient is being viewed through the AR HMD, the motion skeletal model showing a motion of the patient body part based on the improved ROM.

In Example 18, the subject matter of Example 17 includes instructions further causing the processing circuitry to: receive a skeletal model motion pause input; and freeze the motion of the patient body part in the display on the patient AR HMD.

In Example 19, the subject matter of Examples 1-18 includes instructions further causing the processing circuitry to receive a selection of the surgical procedure.

In Example 20, the subject matter of Example 19 includes instructions further causing the processing circuitry to prompt a user for an improved ROM surgical procedure, the improved ROM surgical procedure providing a greater ROM than the surgical procedure.

In Example 21, the subject matter of Examples 1-20 includes instructions further causing the processing circuitry to: receive a skeletal model modification input; generate a modified skeletal model based on the skeletal model modification input; and output the modified skeletal model overlaid on the patient for display on the AR HMD while the patient is being viewed through the AR HMD.

In Example 22, the subject matter of Example 21 includes wherein the skeletal model modification input includes at least a skeletal model joint repositioning, a limb length adjustment, a limb pose adjustment, and a skeletal model reset input.

In Example 23, the subject matter of Examples 12-22 includes instructions further causing the processing circuitry to identify a surgical procedure implication associated with at least one element in the list of surgical procedures associated with the musculoskeletal assessment activity, wherein the output of the selection prompt for the list of surgical procedures includes a display of the surgical procedure implication for display on the AR HMD.

In Example 24, the subject matter of Example 23 includes wherein the surgical procedure implication includes at least one of a recovery time, a recovery physical therapy requirement, and a predicted ROM.

In Example 25, the subject matter of Examples 1-24 includes instructions further causing the processing circuitry to: receive a surgical abstention selection; generate a predicted surgical abstention skeletal model based on the skeletal model, the predicted skeletal model including a degraded ROM based on an abstention from the surgical procedure; and output the predicted surgical abstention skeletal model overlaid on the patient for display on the AR HMD while the patient is being viewed through the AR HMD.

In Example 26, the subject matter of Examples 1-25 includes instructions further causing the processing circuitry to: receive an aging progression input; generate a plurality of aged skeletal models based on the skeletal model, the plurality of aged skeletal models including a plurality of reduced ROM values based on the aging progression input; and output a progression of the plurality of aged skeletal models overlaid on the patient for display on the AR HMD while the patient is being viewed through the AR HMD.

In Example 27, the subject matter of Examples 14-26 includes instructions further causing the processing circuitry to capture a patient motion, wherein the selection of the ROM exercise is based on the patient motion.

Example 28 is a method for augmented reality patient assessment, the method comprising: generating depth sensor data for a patient during a musculoskeletal assessment activity; generating a skeletal model based on the depth sensor data; tracking a patient motion during the musculoskeletal assessment activity; determining a current ROM based on the patient motion; and outputting the current ROM overlaid on the patient for display on an augmented reality (AR) head-mounted display (HMD) while the patient is being viewed through the AR HMD.

In Example 29, the subject matter of Example 28 includes receiving a selection of the musculoskeletal assessment activity; and outputting a description of the musculoskeletal assessment activity for display on the AR HMD while the patient is being viewed through the AR HMD.

In Example 30, the subject matter of Examples 28-29 includes determining a target ROM based on the musculoskeletal assessment activity; and outputting a graphical indication of the target ROM for display on the AR HMD while the patient is being viewed through the AR HMD.

In Example 31, the subject matter of Examples 28-30 includes outputting a guided musculoskeletal activity for display on the AR HMD, the guided musculoskeletal activity providing a patient motion instruction for conducting the musculoskeletal assessment activity.

In Example 32, the subject matter of Examples 28-31 includes receiving motion sensor data from a motion sensor attached to a patient, the motion sensor data characterizing a musculoskeletal motion of the patient; wherein the generation of the skeletal model is further based on the sensor data.

In Example 33, the subject matter of Examples 28-32 includes receiving medical imaging data of a musculoskeletal joint of the patient; wherein the generation of the skeletal model is further based on the medical imaging data.

In Example 34, the subject matter of Examples 28-33 includes receiving a selection of a model surgical procedure; generating a patient procedure model based on the model surgical procedure and the skeletal model; and outputting the patient procedure model for display on the AR HMD while the patient is being viewed through the AR HMD.

In Example 35, the subject matter of Examples 28-34 includes generating a predicted postoperative skeletal model based on the skeletal model, the skeletal model including a preoperative skeletal model, the predicted postoperative skeletal model including an improved range of motion (ROM) based on a surgical procedure; and outputting the predicted postoperative skeletal model overlaid on the patient for display on an augmented reality (AR) head-mounted display (HMD) while the patient is being viewed through the AR HMD.

In Example 36, the subject matter of Example 35 includes generating postoperative depth sensor data for the patient during a postoperative musculoskeletal assessment activity; generating a revised postoperative skeletal model based on the postoperative depth sensor data; and outputting the revised postoperative skeletal model overlaid on the preoperative skeletal model for display on the AR HMD.

In Example 37, the subject matter of Example 36 includes outputting the revised postoperative skeletal model overlaid on the predicted postoperative skeletal model for display on the AR HMD.

In Example 38, the subject matter of Examples 35-37 includes receiving a surgical procedure selection, wherein the predicted postoperative skeletal model is further based on the surgical procedure selection.

In Example 39, the subject matter of Example 38 includes identifying a list of surgical procedures associated with the musculoskeletal assessment activity; and outputting a selection prompt for the list of surgical procedures for display on the AR HMD.

In Example 40, the subject matter of Examples 35-39 includes capturing a plurality of images of the patient, wherein the preoperative skeletal model is further based on the plurality of images of the patient.

In Example 41, the subject matter of Example 40 includes receiving a selection of a ROM exercise; and generating a plurality of ROM images associated with the selected ROM exercise, the plurality of ROM images including the postoperative skeletal model overlaid on the captured images of the patient.

In Example 42, the subject matter of Examples 28-41 includes a patient AR HMD, further including: outputting the predicted skeletal model for display on the AR HMD while the patient is being viewed by a medical practitioner through the AR HMD; capturing an image of the patient as viewed by the medical practitioner through the AR HMD; and outputting the predicted skeletal model overlaid on the image of the patient for display on the patient AR HMD.

In Example 43, the subject matter of Examples 28-42 includes outputting a multiple pose skeletal model, the multiple pose skeletal model configured to display a plurality of positions of a patient body part based on the improved ROM when the multiple pose skeletal model is overlaid on the patient for display on the AR HMD while the patient is being viewed through the AR HMD.

In Example 44, the subject matter of Example 43 includes outputting a motion skeletal model overlaid on the patient for display on the AR HMD while the patient is being viewed through the AR HMD, the motion skeletal model showing a motion of the patient body part based on the improved ROM.

In Example 45, the subject matter of Example 44 includes receiving a skeletal model motion pause input; and freezing the motion of the patient body part in the display on the patient AR HMD.

In Example 46, the subject matter of Examples 28-45 includes receiving a selection of the surgical procedure.

In Example 47, the subject matter of Example 46 includes prompting a user for an improved ROM surgical procedure, the improved ROM surgical procedure providing a greater ROM than the surgical procedure.

In Example 48, the subject matter of Examples 28-47 includes receiving a skeletal model modification input; generating a modified skeletal model based on the skeletal model modification input; and outputting the modified skeletal model overlaid on the patient for display on the AR HMD while the patient is being viewed through the AR HMD.

In Example 49, the subject matter of Example 48 includes wherein the skeletal model modification input includes at least a skeletal model joint repositioning, a limb length adjustment, a limb pose adjustment, and a skeletal model reset input.

In Example 50, the subject matter of Examples 39-49 includes identifying a surgical procedure implication associated with at least one element in the list of surgical procedures associated with the musculoskeletal assessment activity, wherein the output of the selection prompt for the list of surgical procedures includes a display of the surgical procedure implication for display on the AR HMD.

In Example 51, the subject matter of Example 50 includes wherein the surgical procedure implication includes at least one of a recovery time, a recovery physical therapy requirement, and a predicted ROM.

In Example 52, the subject matter of Examples 28-51 includes receiving a surgical abstention selection; generating a predicted surgical abstention skeletal model based on the skeletal model, the predicted skeletal model including a degraded ROM based on an abstention from the surgical procedure; and outputting the predicted surgical abstention skeletal model overlaid on the patient for display on the AR HMD while the patient is being viewed through the AR HMD.

In Example 53, the subject matter of Examples 28-52 includes receiving an aging progression input; generating a plurality of aged skeletal models based on the skeletal model, the plurality of aged skeletal models including a plurality of reduced ROM values based on the aging progression input; and outputting a progression of the plurality of aged skeletal models overlaid on the patient for display on the AR HMD while the patient is being viewed through the AR HMD.

In Example 54, the subject matter of Examples 41-53 includes capturing a patient motion, wherein the selection of the ROM exercise is based on the patient motion.

Example 55 is a non-transitory machine-readable storage medium, comprising instructions that, responsive to being executed with processing circuitry of a computer-controlled device, cause the processing circuitry to: generate depth sensor data for a patient during a musculoskeletal assessment activity; generate a skeletal model based on the depth sensor data; track a patient motion during the musculoskeletal assessment activity; determine a current ROM based on the patient motion; and output the current ROM overlaid on the patient for display on an augmented reality (AR) head-mounted display (HMD) while the patient is being viewed through the AR HMD.

In Example 56, the subject matter of Example 55 includes instructions further causing the processing circuitry to: receive a selection of the musculoskeletal assessment activity; and output a description of the musculoskeletal assessment activity for display on the AR HMD while the patient is being viewed through the AR HMD.

In Example 57, the subject matter of Examples 55-56 includes instructions further causing the processing circuitry to: determine a target ROM based on the musculoskeletal assessment activity; and output a graphical indication of the target ROM for display on the AR HMD while the patient is being viewed through the AR HMD.

In Example 58, the subject matter of Examples 55-57 includes instructions further causing the processing circuitry to output a guided musculoskeletal activity for display on the AR HMD, the guided musculoskeletal activity providing a patient motion instruction for conducting the musculoskeletal assessment activity.

In Example 59, the subject matter of Examples 55-58 includes instructions further causing the processing circuitry to receive motion sensor data from a motion sensor attached to a patient, the motion sensor data characterizing a musculoskeletal motion of the patient; wherein the generation of the skeletal model is further based on the sensor data.

In Example 60, the subject matter of Examples 55-59 includes instructions further causing the processing circuitry to receive medical imaging data of a musculoskeletal joint of the patient; wherein the generation of the skeletal model is further based on the medical imaging data.

In Example 61, the subject matter of Examples 55-60 includes instructions further causing the processing circuitry to: receive a selection of a model surgical procedure; generate a patient procedure model based on the model surgical procedure and the skeletal model; and output the patient procedure model for display on the AR HMD while the patient is being viewed through the AR HMD.

In Example 62, the subject matter of Examples 55-61 includes instructions further causing the processing circuitry to: generate a predicted postoperative skeletal model based on the skeletal model, the skeletal model including a preoperative skeletal model, the predicted postoperative skeletal model including an improved range of motion (ROM) based on a surgical procedure; and output the predicted postoperative skeletal model overlaid on the patient for display on an augmented reality (AR) head-mounted display (HMD) while the patient is being viewed through the AR HMD.

In Example 63, the subject matter of Example 62 includes instructions further causing the processing circuitry to: generate postoperative depth sensor data for the patient during a postoperative musculoskeletal assessment activity; generate a revised postoperative skeletal model based on the postoperative depth sensor data; and output the revised postoperative skeletal model overlaid on the preoperative skeletal model for display on the AR HMD.

In Example 64, the subject matter of Example 63 includes instructions further causing the processing circuitry to output the revised postoperative skeletal model overlaid on the predicted postoperative skeletal model for display on the AR HMD.

In Example 65, the subject matter of Examples 62-64 includes instructions further causing the processing circuitry to receive a surgical procedure selection, wherein the predicted postoperative skeletal model is further based on the surgical procedure selection.

In Example 66, the subject matter of Example 65 includes instructions further causing the processing circuitry to: identify a list of surgical procedures associated with the musculoskeletal assessment activity; and output a selection prompt for the list of surgical procedures for display on the AR HMD.

In Example 67, the subject matter of Examples 55-66 includes instructions further causing the processing circuitry to a patient AR HMD, the instructions further causing the processing circuitry to: output the predicted skeletal model for display on the AR HMD while the patient is being viewed by a medical practitioner through the AR HMD; capture an image of the patient as viewed by the medical practitioner through the AR HMD; and output the predicted skeletal model overlaid on the image of the patient for display on the patient AR HMD.

In Example 68, the subject matter of Examples 55-67 includes instructions further causing the processing circuitry to output a multiple pose skeletal model, the multiple pose skeletal model configured to display a plurality of positions of a patient body part based on the improved ROM when the multiple pose skeletal model is overlaid on the patient for display on the AR HMD while the patient is being viewed through the AR HMD.

In Example 69, the subject matter of Example 68 includes instructions further causing the processing circuitry to output a motion skeletal model overlaid on the patient for display on the AR HMD while the patient is being viewed through the AR HMD, the motion skeletal model showing a motion of the patient body part based on the improved ROM.

In Example 70, the subject matter of Example 69 includes instructions further causing the processing circuitry to: receive a skeletal model motion pause input; and freeze the motion of the patient body part in the display on the patient AR HMD.

In Example 71, the subject matter of Examples 55-70 includes instructions further causing the processing circuitry to receive a selection of the surgical procedure.

In Example 72, the subject matter of Example 71 includes instructions further causing the processing circuitry to prompt a user for an improved ROM surgical procedure, the improved ROM surgical procedure providing a greater ROM than the surgical procedure.

In Example 73, the subject matter of Examples 55-72 includes instructions further causing the processing circuitry to: receive a skeletal model modification input; generate a modified skeletal model based on the skeletal model modification input; and output the modified skeletal model overlaid on the patient for display on the AR HMD while the patient is being viewed through the AR HMD.

In Example 74, the subject matter of Example 73 includes wherein the skeletal model modification input includes at least a skeletal model joint repositioning, a limb length adjustment, a limb pose adjustment, and a skeletal model reset input.

In Example 75, the subject matter of Examples 66-74 includes instructions further causing the processing circuitry to identify a surgical procedure implication associated with at least one element in the list of surgical procedures associated with the musculoskeletal assessment activity, wherein the output of the selection prompt for the list of surgical procedures includes a display of the surgical procedure implication for display on the AR HMD.

In Example 76, the subject matter of Example 75 includes wherein the surgical procedure implication includes at least one of a recovery time, a recovery physical therapy requirement, and a predicted ROM.

In Example 77, the subject matter of Examples 55-76 includes instructions further causing the processing circuitry to: receive a surgical abstention selection; generate a predicted surgical abstention skeletal model based on the skeletal model, the predicted skeletal model including a degraded ROM based on an abstention from the surgical procedure; and output the predicted surgical abstention skeletal model overlaid on the patient for display on the AR HMD while the patient is being viewed through the AR HMD.

In Example 78, the subject matter of Examples 55-77 includes instructions further causing the processing circuitry to: receive an aging progression input; generate a plurality of aged skeletal models based on the skeletal model, the plurality of aged skeletal models including a plurality of reduced ROM values based on the aging progression input; and output a progression of the plurality of aged skeletal models overlaid on the patient for display on the AR HMD while the patient is being viewed through the AR HMD.

In Example 79, the subject matter of Example undefined includes instructions further causing the processing circuitry to capture a patient motion, wherein the selection of the ROM exercise is based on the patient motion.

Example 80 is a system for patient assessment, the system comprising: a depth sensor to generate depth sensor data for a patient; an image sensor to capture a plurality of images of the patient; processing circuitry; and a memory that includes instructions, the instructions, when executed by the processing circuitry, cause the processing circuitry to: generate a preoperative skeletal model based on the depth sensor data; generate a postoperative skeletal model based on the preoperative skeletal model, the postoperative skeletal model including an improved range of motion based on a predetermined surgical procedure; and outputting for display the postoperative skeletal model overlaid on the plurality of images of the patient.

Example 81 is a method for assessment, the method comprising: generating depth sensor data for a patient; capturing a plurality of images of the patient; generating a preoperative skeletal model based on the depth sensor data; generating a postoperative skeletal model based on the preoperative skeletal model, the postoperative skeletal model including an improved range of motion based on a predetermined surgical procedure; and outputting for display the postoperative skeletal model overlaid on the plurality of images of the patient.

Example 82 is at least one machine-readable medium including instructions that, when executed by processing circuitry, cause the processing circuitry to perform operations to implement of any of Examples 1-81.

Example 83 is an apparatus comprising means to implement of any of Examples 1-81.

Example 84 is a system to implement of any of Examples 1-81.

Example 85 is a method to implement of any of Examples 1-81.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A system for augmented reality patient assessment, the system comprising:
an augmented reality (AR) head-mounted display (HMD);
a depth sensor to generate depth sensor data for a patient during a musculoskeletal assessment activity;
processing circuitry; and
a memory that includes instructions, the instructions, when executed by the processing circuitry, cause the processing circuitry to:
generate a skeletal model based on the depth sensor data;
track a patient motion during the musculoskeletal assessment activity;
determine a current ROM based on the patient motion; and
output the current ROM overlaid on the patient for display on the AR HMD while the patient is being viewed through the AR HMD.

2. The system of claim 1, the instructions further causing the processing circuitry to:
receive a selection of the musculoskeletal assessment activity; and
output a description of the musculoskeletal assessment activity for display on the AR HMD while the patient is being viewed through the AR HMD.

3. The system of claim 1, the instructions further causing the processing circuitry to:
determine a target ROM based on the musculoskeletal assessment activity; and
output a graphical indication of the target ROM for display on the AR HMD while the patient is being viewed through the AR HMD.

4. The system of claim 1, the instructions further causing the processing circuitry to output a guided musculoskeletal activity for display on the AR HMD, the guided musculoskeletal activity providing a patient motion instruction for conducting the musculoskeletal assessment activity.

5. The system of claim 1, the instructions further causing the processing circuitry to receive motion sensor data from a motion sensor attached to a patient, the motion sensor data characterizing a musculoskeletal motion of the patient;
wherein the generation of the skeletal model is further based on the sensor data.

6. The system of claim 1, the instructions further causing the processing circuitry to receive medical imaging data of a musculoskeletal joint of the patient;
wherein the generation of the skeletal model is further based on the medical imaging data.

7. The system of claim 1, the instructions further causing the processing circuitry to:
receive a selection of a model surgical procedure;
generate a patient procedure model based on the model surgical procedure and the skeletal model; and
output the patient procedure model for display on the AR HMD while the patient is being viewed through the AR HMD.

8. The system of claim 1, the instructions further causing the processing circuitry to:
generate a predicted postoperative skeletal model based on the skeletal model, the skeletal model including a preoperative skeletal model, the predicted postoperative skeletal model including an improved range of motion (ROM) based on a surgical procedure; and
output the predicted postoperative skeletal model overlaid on the patient for display on the AR HMD while the patient is being viewed through the AR HMD.

9. The system of claim 8, the depth sensor further to generate postoperative depth sensor data for the patient during a postoperative musculoskeletal assessment activity; and
the instructions further causing the processing circuitry to:
generate a revised postoperative skeletal model based on the postoperative depth sensor data; and
output the revised postoperative skeletal model overlaid on the preoperative skeletal model for display on the AR HMD.

10. The system of claim 8, the instructions further causing the processing circuitry to receive a surgical procedure selection, wherein the predicted postoperative skeletal model is further based on the surgical procedure selection.

11. The system of claim 10, the instructions further causing the processing circuitry to:
identify a list of surgical procedures associated with the musculoskeletal assessment activity; and
output a selection prompt for the list of surgical procedures for display on the AR HMD.

12. A method for augmented reality patient assessment, the method comprising:
generating depth sensor data for a patient during a musculoskeletal assessment activity;
generating a skeletal model based on the depth sensor data;
tracking a patient motion during the musculoskeletal assessment activity;
determining a current ROM based on the patient motion; and
outputting the current ROM overlaid on the patient for display on an augmented reality (AR) head-mounted display (HMD) while the patient is being viewed through the AR HMD.

13. The method of claim 12, further including:
receiving a selection of the musculoskeletal assessment activity; and
outputting a description of the musculoskeletal assessment activity for display on the AR HMD while the patient is being viewed through the AR HMD.

14. The method of claim 12, further including:
determining a target ROM based on the musculoskeletal assessment activity; and
outputting a graphical indication of the target ROM for display on the AR HMD while the patient is being viewed through the AR HMD.

15. The method of claim 12, further including outputting a guided musculoskeletal activity for display on the AR HMD, the guided musculoskeletal activity providing a patient motion instruction for conducting the musculoskeletal assessment activity.

16. The method of claim 12, further including receiving motion sensor data from a motion sensor attached to a patient, the motion sensor data characterizing a musculoskeletal motion of the patient;
wherein the generation of the skeletal model is further based on the sensor data.

17. The method of claim 12, further including receiving medical imaging data of a musculoskeletal joint of the patient;
wherein the generation of the skeletal model is further based on the medical imaging data.

18. The method of claim 12, further including:
receiving a selection of a model surgical procedure;
generating a patient procedure model based on the model surgical procedure and the skeletal model; and
outputting the patient procedure model for display on the AR HMD while the patient is being viewed through the AR HMD.

19. The method of claim 12, further including:
generating a predicted postoperative skeletal model based on the skeletal model, the skeletal model including a preoperative skeletal model, the predicted postoperative skeletal model including an improved range of motion (ROM) based on a surgical procedure; and
outputting the predicted postoperative skeletal model overlaid on the patient for display on an augmented reality (AR) head-mounted display (HMD) while the patient is being viewed through the AR HMD.

20. The method of claim 19, further including:
generating postoperative depth sensor data for the patient during a postoperative musculoskeletal assessment activity;
generating a revised postoperative skeletal model based on the postoperative depth sensor data; and
outputting the revised postoperative skeletal model overlaid on the preoperative skeletal model for display on the AR HMD.

21. The method of claim 19, further including receiving a surgical procedure selection, wherein the predicted postoperative skeletal model is further based on the surgical procedure selection.

22. The method of claim 21, further including:
identifying a list of surgical procedures associated with the musculoskeletal assessment activity; and
outputting a selection prompt for the list of surgical procedures for display on the AR HMD.

23. A non-transitory machine-readable storage medium, comprising instructions that, responsive to being executed with processing circuitry of a computer-controlled device, cause the processing circuitry to:
generate depth sensor data for a patient during a musculoskeletal assessment activity;
generate a skeletal model based on the depth sensor data;
track a patient motion during the musculoskeletal assessment activity;
determine a current ROM based on the patient motion; and
output the current ROM overlaid on the patient for display on an augmented reality (AR) head-mounted display (HMD) while the patient is being viewed through the AR HMD.

24. The non-transitory machine-readable storage medium of claim 23, the instructions further causing the processing circuitry to:
receive a selection of the musculoskeletal assessment activity; and
output a description of the musculoskeletal assessment activity for display on the AR HMD while the patient is being viewed through the AR HMD.

25. The non-transitory machine-readable storage medium of claim 23, the instructions further causing the processing circuitry to:
determine a target ROM based on the musculoskeletal assessment activity; and
output a graphical indication of the target ROM for display on the AR HMD while the patient is being viewed through the AR HMD.
